SEQUENCE LISTING (3 sequences)

SEQ ID NO: 1 - DNA, 12 bp, Artificial Sequence
  Description of Artificial Sequence  alpha site
ttcctcgggaat

SEQ ID NO: 2 - DNA, 12 bp, Artificial Sequence
  Description of Artificial Sequence  beta site
aaactcgagaga

SEQ ID NO: 3 - DNA, 6 bp, Artificial Sequence
  Description of Artificial Sequence binding site
cncgng

(19) United States
(12) Patent Application Publication
(10) Pub. No.: US 2003/0108935 A1
(43) Pub. Date: Jun. 12, 2003

(54) Probing binding interactions between molecules and nucleic acids by unzipping a nucleic acid molecule double helix

(76) Inventors: Michelle D. Wang, Ithaca, NY (US); Steven J. Koch, Ithaca, NY (US)

Correspondence Address: Michael L. Goldman, Esq., NIXON PEABODY LLP, Clinton Square, P.O. Box 31051, Rochester, NY, 14603-1051, US

(21) Appl. No.: 10/280,359

(22) Filed: Oct. 24, 2002

Related U.S. Application Data
(60) Provisional application No. 60/335,318, filed on Oct. 24, 2001.

(51) Int. Cl. C12Q 1/68; C12P 19/34

(52) U.S. Cl. 435/6; 435/91.2

(57) ABSTRACT

The present invention is directed to a method of identifying the location of a binding site for a binding molecule on a double-stranded nucleic acid molecule, a method of directly determining the equilibrium association constant of a target binding molecule specific to a double-stranded nucleic acid molecule, a method of determining the dynamic force signature of a target binding molecule in relation to a binding site on a double-stranded nucleic acid molecule, a method of identifying whether a target nucleic acid molecule is present in a sample, a method of producing a restriction map, or a method of identifying whether a target protein is present in a sample. The methods are carried out by comparing the force required to unzip the first and second nucleic acid strands of the double-stranded nucleic acid molecule without and (potentially) with a binding molecule bound to the double-stranded nucleic acid molecule.

This invention was developed with government funding by the National Institutes of Health NIH Grant No. 08-RIGM59849A and the National Institutes of Health Molecular Biophysics Training Grant No. T32-GM08267. The U.S. Government may have certain rights.

This application claims the benefit of U.S. Provisional Patent Application Serial No. 60/335,318, filed Oct. 24, 2001.

### FIELD OF THE INVENTION

The present invention relates to the analysis of molecule-nucleic acid interactions by unzipping a nucleic acid double helix.

### BACKGROUND OF THE INVENTION

Protein-DNA interactions are essential to cellular processes. In replication, transcription, recombination, DNA repair, and DNA packaging, proteins bind to DNA as activators or repressors, to recruit other proteins, or to carry out various catalytic activities. These DNA-binding proteins include polymerases, helicases, nucleases, isomerases, ligases, histones, and others. Because of their great importance, protein-DNA interactions have justifiably drawn much attention from biochemical researchers over the last half-century. More recently, the application of single-molecule mechanical techniques to the interactions of proteins and DNA has attracted great interest, in particular for the study of molecular motors such as RNA polymerases, DNA polymerases, helicases, and topoisomerases (Yin et al., “Transcription Against an Applied Force,” Science 270:1653-1657 (1995); Wang et al., “Force and Velocity Measured for Single Molecules of RNA Polymerase,” Science 282:902-907 (1998); Wuite et al., “Single-Molecule Studies of the Effect of Template Tension on T7 DNA Polymerase Activity,” Nature 404:103-106 (2000); Bianco et al., “Processive Translocation and DNA Unwinding by Individual RecBCD Enzyme Molecules,” Nature 409:374-378 (2001); Dohoney et al., “Chi-Sequence Recognition and DNA Translocation by Single RecBCD Helicase/Nuclease Molecules,” Nature 409:370-374 (2001); Strick et al., “Single-Molecule Analysis of DNA Uncoiling by a Type II Topoisomerase,” Nature 404:901-904 (2000)), as well as the investigation of chromatin structure (Cui et al., “Pulling a Single Chromatin Fiber Reveals the Forces that Maintain Its Higher-Order Structure,” Proc. Natl. Acad. Sci. USA 97:127-132 (2000); Bennink et al., “Unfolding Individual Nucleosomes by Stretching Single Chromatin Fibers with Optical Tweezers,” Nat. Struct. Biol. 8:606-610 (2001); Brower-Toland et al., “Mechanical Disruption of Individual Nucleosomes Reveals a Reversible Multistage Release of DNA,” Proc. Natl. Acad. Sci. USA 99:1960-1965 (2002)).

Critical parameters for protein-DNA interactions include location, specificity, and strength of interaction. Many biochemical techniques exist which provide information about these parameters, but none provide all of them at once on a molecule-by-molecule basis.

Previously, it was demonstrated that the force required to unzip naked DNA depends strongly on the local nucleotide sequence (Bockelmann et al., “DNA Strand Separation Studied by Single Molecule Force Measurements,” Phys. Rev. E. 58:2386-2394 (1998); Bockelmann et al., “Molecular Stick-Slip Motion Revealed by Opening DNA with Piconewton Forces,” Phys. Rev. Lett. 79:4489-4492 (1997); Essevaz-Roulet et al., “Mechanical Separation of the Complementary Strands of DNA,” Proc. Natl. Acad. Sci. USA 94:11935-11940 (1997)). Furthermore, this force could be predicted from a simple quasi-equilibrium model accounting only for the energies of A-T versus G-C base pairs and the series compliance of the system.

The recent technical advances in the manipulation of single biomolecules have made it possible to mechanically disrupt the bonds of interacting biomolecules. More recent theoretical development of dynamic force spectroscopy (DFS) by Evans et al. (Evans et al., “Dynamic Strength of Molecular Adhesion Bonds,” Biophys. J. 72:1541-1555 (1997); Evans et al., “Probing the Relation Between Force—Lifetime—and Chemistry in Single Molecular Bonds,” Annu. Rev. Biophys. Biomol. Struct. 30:105-128 (2001)) has made mechanical disruption a viable method of quantitatively investigating the underlying energetics of interacting biomolecules. DFS has been applied to protein-ligand interactions (Merkel et al., “Energy Landscapes of Receptor-Ligand Bonds Explored with Dynamic Force Spectroscopy,” Nature 397:50-53 (1999) (Avidin-biotin)), membrane receptor-ligand interactions (Prechtel et al., “Dynamic Force Spectroscopy to Probe Adhesion Strength of Living Cells,” Phys. Rev. Lett. 89:028101 (2002)), membrane protein—membrane protein interactions (Zhang et al., “Force Spectroscopy of the Leukocyte Function-Associated Antigen-1/Intercellular Adhesion Molecule-1 Interaction,” Biophys. J. 83:2270-2279 (2002)), protein-protein interactions (Fritz et al., “Force-Mediated Kinetics of Single P-Selectin/Ligand Complexes Observed by Atomic Force Microscopy,” Proc. Natl. Acad. Sci. USA 95:12283-12288 (1998) (P-selectin/ligand); Rief et al., “Reversible Unfolding of Individual Titin Immunoglobulin Domains by AFM,” Science 276:1109-1112 (1997) (Titin unfolding)), and protein-DNA interactions (Brower-Toland et al., “Mechanical Disruption of Individual Nucleosomes Reveals a Reversible Multistage Release of DNA,” Proc. Natl. Acad. Sci. USA 99:1960-1965 (2002)). Despite the importance of protein-DNA interactions the application of DFS to protein-DNA interactions has so far been limited to the recent report of the disruption of nucleosomal DNA (Brower-Toland et al., “Mechanical Disruption of Individual Nucleosomes Reveals a Reversible Multistage Release of DNA,” Proc. Natl. Acad. Sci. USA 99:1960-1965 (2002)).

The need remains in the art to evaluate protein-nucleic acid molecule interactions. The present invention is directed to satisfying this need in the art.

### SUMMARY OF THE INVENTION

One aspect of the present invention is directed to a method of identifying the location of a binding site for a binding molecule on a double-stranded nucleic acid molecule. A double-stranded nucleic acid molecule having a first nucleic acid strand and a second nucleic acid strand is provided. In addition, a binding molecule is provided. The double-stranded nucleic acid molecule and the binding molecule are brought into contact under conditions effective to yield a modified nucleic acid molecule, including the binding molecule bound to the double-stranded nucleic acid molecule. The first and second nucleic acid strands are unzipped from one another under conditions effective to disrupt the binding molecule, if any, from being bound to the double-stranded nucleic acid molecule. Any binding site locations for a given binding molecule on the nucleic acid molecule is identified by comparing the force required to unzip the first and second nucleic acid strands from one another with the force required to unzip the nucleic acid molecule with no binding molecule bound thereto. Locations with a change in the force required to unzip the modified nucleic acid molecule compared to the force required to unzip the nucleic acid molecule with no binding molecule bound thereto are binding sites on the modified nucleic acid molecule.

Another aspect of the present invention is directed to a method of directly determining an equilibrium association constant of a target binding molecule specific to a double-stranded nucleic acid molecule. This involves providing a double-stranded nucleic acid molecule suspected of having a binding site for the target binding molecule, where the double-stranded nucleic acid molecule has both a first nucleic acid strand and a second nucleic acid strand. The target binding molecule is also provided. The double-stranded nucleic acid molecule and the target binding molecule are contacted with one another under conditions effective to yield a modified nucleic acid molecule. The modified nucleic acid molecule includes the target binding molecule bound to the double-stranded nucleic acid molecule. The first and second nucleic acid strands are unzipped from one another under conditions effective to disrupt the target binding molecule, if any, bound to the double-stranded nucleic acid molecule. A determination is then made as to whether a binding site on the double stranded nucleic acid molecule is occupied by the target binding molecule. This is done by comparing the force required to unzip the first and second nucleic acid strands from one another with the force required to unzip the double-stranded nucleic acid molecule having no target binding molecule bound to the double-stranded nucleic acid molecule. A change in force required to unzip the double-stranded nucleic acid molecule having no target binding molecule indicates the presence of a target binding molecule. The above steps (as described in this paragraph) are repeated at least one time, and the data is used to calculate the ratio of occupied to unoccupied binding sites as a result of carrying out the above steps of the method of the present invention. The resulting ratio is divided by the bulk protein concentrations of bound to unbound target binding molecules to the suspected binding site based on the determination described above relating to change of force required to unzip the double-stranded nucleic acid molecule. This ratio is the equilibrium constant of the target binding molecule to the suspected binding site.

Another aspect of the present invention is directed to a method of determining the dynamic force signature of a target binding molecule in relation to a binding site on a double-stranded nucleic acid molecule. This involves providing a double-stranded nucleic acid molecule suspected of having a binding site for the target binding molecule, where the double-stranded nucleic acid molecule has both a first nucleic acid strand and second nucleic acid strand. The double-stranded nucleic acid molecule and the target binding molecule are contacted with one another under conditions effective to yield a modified nucleic acid molecule having at least one binding complex (i.e., a complex including the target binding molecule bound to the double-stranded nucleic acid molecule). The first and second nucleic acid strands are unzipped from one another under conditions effective to disrupt the target binding molecule, if any, bound to the double-stranded nucleic acid molecule. A determination is made as to the unzipping disruption location, starting force, peak force for disruption, and force loading pattern for the target binding molecule. The previous steps of this paragraph are repeated, and, thereafter, the dynamic force signature for the binding molecule are calculated based on the unzipping location, the starting force, the peak force, and the force loading pattern determinations.

Another aspect of the present invention is directed to a method of identifying whether a target nucleic acid molecule is present in a sample. This involves providing a sample potentially containing a double-stranded target nucleic acid molecule having a first nucleic acid strand and a second nucleic acid strand. A protein which binds to a binding site on the target nucleic acid molecule is provided. The sample and the protein are contacted with one another under conditions effective to permit the protein to bind to the target nucleic acid molecule, if present in the sample. The first and second nucleic acid strands are unzipped from one another under conditions effective to separate the two strands. Thereafter, the presence of any target nucleic acid molecules is identified by measuring the force required to unzip the first and second nucleic acid strands from one another, where a change in force indicates the presence of the target nucleic acid molecule in the sample.

An additional aspect of the present invention is directed to a method of producing a restriction map for a nucleic acid molecule. This involves providing a double-stranded nucleic acid molecule having a first nucleic acid strand and a second nucleic acid strand. A restriction endonuclease is also provided. The double-stranded nucleic acid molecule and the restriction endonuclease are contacted with one another under conditions effective to yield a binding complex of the restriction endonuclease bound to the double-stranded nucleic acid molecule, while prohibiting the restriction endonuclease from cutting the nucleic acid molecule. The first and second nucleic acid strands are unzipped from one another under conditions effective to separate the first and second nucleic acid strands from one another. Thereafter, the binding site locations are identified, if any, for the restriction endonuclease on the nucleic acid molecule. This is done by comparing the force required to unzip the first and second nucleic acid strands from one another with the force required to unzip the double-stranded nucleic acid molecule having no restriction endonuclease bound thereto, where a change in force required to unzip the double-stranded nucleic acid molecule having no restriction endonuclease indicates the location of a binding site for the restriction endonuclease on the double-stranded nucleic acid molecule.

Yet another aspect of the present invention is directed to a method of identifying whether a target protein is present in a sample. This involves providing a sample potentially containing a target protein and providing a double-stranded nucleic acid molecule. The double-stranded nucleic acid molecule has a first nucleic acid strand and a second nucleic acid strand, as well as a binding site to which the target protein binds. The sample and the double-stranded nucleic acid molecule are contacted with one another under conditions effective to permit the target protein, if present, to bind to the double-stranded nucleic acid molecule. The first and second nucleic acid strands are unzipped under conditions effective to separate the first and second nucleic acid strands from one another. Thereafter, the presence of the target protein is identified by comparing the force required to unzip the first and second nucleic acid strands from one another with the force required to unzip the double-stranded nucleic acid molecule having no target protein bound thereto, where a change in force required to unzip the double-stranded nucleic acid molecule having no target protein indicates the presence the target protein in the sample.

The methods of the present invention, including the unzipping force analysis of protein association (“UFAPA”) technique, are general tools for detection of binding molecule/double-stranded nucleic acid interactions, including protein-DNA interactions. It is a single nucleic acid molecule technique that yields the locations of bound proteins, the equilibrium association constants for the binding molecule-DNA interactions, and dynamic force signatures of the binding. Because the method of the present invention is a single molecule technique, only a small amount of nucleic acids (e.g., DNA, RNA) and the binding molecule (e.g., proteins) are required to make measurements. This is a great advantage over the bulk studies common in the art. Furthermore, since the unzipped nucleic acid molecule can be reversibly annealed, the same nucleic acid molecule can be used repetitively in the methods of the present invention. Therefore, the methods of the present invention have the potential to map the affinity of multiple molecules on a single nucleic acid molecule.

The methods of the present invention also allows for exceptionally good spatial resolution. For example, over a 4 kilobasepair (kb) DNA molecule, the locations of protein binding sites can be determined with an accuracy of 25 basepairs (bp), a precision of 30 bp, and a relative location determination of 3 bp. This resolution is not expected to decrease appreciably for much longer DNA molecules (for example, DNA molecules of a few Mbp). This resolution is unmatched by most traditional biochemical techniques, except for footprinting. However, footprinting is limited to smaller pieces of DNA (i.e., of ˜1000 bp or less).

The methods of the present invention are also advantageous due to their speed of use. For example, UFAPA is a very fast method once the DNA molecule is constructed for unzipping. The detection rate is better than 2 kb/second (s) over a 4 bp DNA molecule. This rate is clearly unmatched by any other biochemical techniques.

The methods of the present invention also give direct measurements of equilibrium association constants for protein-DNA interactions. Traditional biochemical methods of measurement of equilibrium association constants are complicated by non-specific binding of proteins to DNA and the possibility of detachment of proteins before a measurement is obtained (for example, while the sample is entering a gel). However, UFAPA does not suffer from these problems since it is specific and fast.

In addition to measuring equilibrium association constants for molecule-nucleic acid (e.g., protein-DNA) interactions, the methods of the present invention may be used to generate dynamic signatures for a given binding molecule (e.g., proteins) and DNA interactions. This feature allows further differentiation of protein-DNA interactions even when they have identical binding sites and association constants.

The methods of the present invention also have potential for a large number of biotechnological applications. For example, UFAPA could be used for restriction mapping as presented in Koch et al., “Probing Protein-DNA Interactions by Unzipping a Single DNA Double Helix,” Biophys. J. 83:1098-1105 (2002), the entire disclosure of which is hereby incorporated by reference in its entirety. This is an important application of UFAPA since restriction mapping is the first critical step in genomic sequencing. The methods of the present invention can also be used for DNA sequencing, because the sequence of DNA may be determined with a large number of restriction enzymes. UFAPA could be used to locate binding sites of a DNA binding protein whose binding sites are yet unknown. This could be a very useful step before the detailed biochemical footprinting begins. Once the binding sites are located, the binding sequence could be used for further/easier/cheaper purification of the protein, for example, on an affinity column which uses this sequence. Similarly, it could be used to detect new DNA binding proteins for binding to specific sequences. It may be possible to use UFAPA to distinguish between different forms of a bound protein (e.g., phosphorylated or un-phosphorylated, methylated or unmethylated, acetylated or unacetylated). This may be especially important for the understanding of gene regulation. UFAPA may also help distinguish between various strains of a given binding molecule, based on changes in the dynamic force signature. Further, naked DNA mapping or protein UFAPA mapping could be quite useful for mapping difficult regions of the genome of an organism, including, for example, humans and other important species. The direct mapping of UFAPA should avoid some of the problems encountered in highly repetitive areas of genomes.

The methods of the present invention could be used to rapidly assay the binding affinities and strengths of molecules specifically designed to bind to specific sequences of DNA for therapeutic purposes (e.g., to block transcription of harmful genes). Also, UFAPA could help develop therapies by measuring reduction in affinities. It may also be possible to extend UFAPA to specific assays testing for the presence of DNA-binding disease related molecules, such as reverse transcriptases.

### BRIEF DESCRIPTION OF THE DRAWINGS

FIGS. 1A-1B show the experimental configuration of an embodiment of the present invention. FIG. 1A is a schematic drawing of the unzipping configuration. The two strands of the DNA molecule are unzipped with a feedback-enhanced optical trap. Unzipping proceeds rather smoothly until a DNA-bound protein is encountered, and additional force is required to unzip through it. The location of the unzipping fork is indicated by an unzipping index j, which is the number of base pairs unzipped from the coverslip bound end of the DNA molecule. FIG. 1B is a schematic of the DNA molecule, not to scale. The complete sequences are shown for the two oligonucleotides comprising the insert duplex. The locations of the digoxigenin and biotin labels, and the nick are shown.

FIGS. 2A-2B shows a comparison of DNA unzipping data in the absence and presence of binding proteins. FIG. 2A is a graphical representation of the force versus extension for two identical DNA molecules unzipped in the absence (black lines) or presence (red lines) of BsoBI (700 pM) using a velocity clamp at 700 nm/s. The resistance to unzipping by BsoBI resulted in distinctive peaks that were not present with the naked DNA. The dotted black curves represent calculated force-extension relations for DNA molecules as shown in FIG. 1A, where j is set to the starting index for each of the BsoBI binding sites. FIG. 2B is a graphical representation of the unzipping index j vs. time. The unzipping index j was calculated from data shown in FIG. 2A. The origin of the time axis is arbitrary. Horizontal dashed lines indicate the expected binding sites, corresponding to the dotted curves in FIG. 2A. A peak in FIG. 2A that resulted from BsoBI resistance became a large plateau because the unzipping index j remained unchanged until BsoBI unbound. At the concentration of BsoBI used, some sites remain unoccupied, as shown at sites 1400 bp, 3100 bp, 3500 bp, and 4000 bp (indicated by horizontal arrows).

FIGS. 3A-3B show detection of protein binding sites. FIG. 3A is a graphical representation of force vs. unzipping index j in the presence of EcoRI ([EcoRI]=83 pM, [Na+]=131 mM) using a velocity clamp at 280 nm/s. The resistance to unzipping by EcoRI resulted in distinctive peaks at the locations of bound EcoRI. Each peak was followed by a sudden reduction of force after EcoRI unbound. FIG. 3B is a graphical representation of the dwell time vs. unzipping index j for forces >20 pN (threshold force for inclusion of data). The vertical dashed lines indicate peaks in the dwell time distributions. The vertical bars below the unzipping index axis indicate the predicted binding locations of EcoRI based on the known recognition sequence on the pCP681-derived construct.

FIG. 4 shows non-catalytic restriction mapping. This is a summary of data from multiple DNA molecules for the three restriction enzymes studied. For each enzyme, red bars mark the expected recognition sites. For BsoBI, α and β represent two different canonical binding sites. The gray-scale intensity represents the binding frequency in log scale determined from unzipping experiments. Data for BsoBI ([BsoBI]=700 pM; 7 DNA molecules unzipped) and XhoI ([XhoI]=4.6 nM; 4 DNA molecules unzipped) are for binding to the repetitive DNA molecule, while data for EcoRI ([EcoRI]=300 pM; 16 DNA molecules unzipped) are for binding to the pBR322-derived DNA molecule, for which there is one well-known binding site.

FIGS. 5A-5B show Na+ concentration dependence of the equilibrium association constant for EcoRI binding to its site on pBR322, plotted on a log-log scale. (A) Solid circles represent measured association constant, K_{A}, as described in text (23° C., 10 mM Hepes, pH 7.6, variable Na+ concentration). The open circle represents filter binding data for binding to the pBR322 at 21.1° C. (10 mM Hepes, pH 7.6 at 20° C., NaCl added to Na+ concentration of 100 mM) (Ha et al., “Role of the Hydrophobic Effect in Stability of Site-Specific Protein-DNA Complexes,” J. Mol. Biol. 209:801-816 (1989), the entire disclosure of which is hereby incorporated by reference in its entirety). Open squares represent filter binding data for binding to the pBR322 site at 37° C. (20 mM Tris.HCl, pH 7.6, variable Na+ concentration (Terry et al., “Thermodynamic Parameters Governing Interaction of EcoRI Endonuclease with Specific and Nonspecific DNA Sequences,” J. Biol. Chem. 258:9820-9825 (1983), the entire disclosure of which is hereby incorporated by reference in its entirety). FIG. 5B shows the tabulation of the UFAPA data from FIG. 5A. N represents number of DNA molecules probed for binding, and n represents number of binding sites found to be occupied. [EcoRI] represents the actual EcoRI concentration used in pM.

FIG. 6 is a schematic of the unzipping configuration (not to scale). Applied tension unzips the two strands of the DNA molecule. The presence of a DNA-binding protein causes a resistance to unzipping, observed as an increase in the force required to separate the strands at that location. The location of the unzipping fork is indicated by an unzipping index j.

FIGS. 7A-7B show a graphical demonstration of loading rate clamped unzipping data for BsoBI disruption from the pCP681 derived DNA molecule. By calculating the length of ssDNA in real time, and implementing a calibrated freely-jointed chain polymer model, the rate of stretching is modulated to keep the loading rate independent of polymer length or force value. FIG. 7A shows the force time series and demonstrates the uniform force loading rate for forces greater than 15 pN. FIG. 7B shows the calculated unzipping index, j, versus time. The horizontal steps represent data where the restriction enzyme pins the unzipping index at a certain value until the complex disrupts.

FIG. 8 is a graphical representation of the dynamic force spectroscopy for BsoBI α sites (N=243 events total). Bars represent unbinding forces in 5 pN width bins, grouped by common force loading rate. Solid lines represent predicted PDFs resulting from a global maximum likelihood fit (two parameters) of all the data. Moving from the bottom graph to the top, force loading rates are 6 pN/s, 12 pN/s, 23 pN/s, 58 pN/s, 230 pN/s, 600 pN/s, 1100 pN/s.

FIG. 9 is a graphical representation of the dynamic force spectroscopy for three binding species. Open circles represent BsoBI unbinding from a sites (ttcCTCGGGaat) (SEQ ID NO: 1), Open squares represent BsoBI unbinding from β sites (aaaCTCGAGaga) (SEQ ID NO:2), and filled squares represent XhoI unbinding from β sites. Data points represent most-likely unbinding rate for a given stretch rate, obtained from sliding-window histograms. Error bars represent standard deviation of sliding window histograms to 1,000 monte-carlo simulations using the particular loading rate, and the global fit parameters. Solid lines represent the predicted most likely forces, based on the overall maximum likelihood fits, while dashed lines are simple linear fits to the data, as would traditionally be done in DFS.

FIG. 10 is a graphical representation showing the distinct unbinding signatures. Solid gray bars represent BsoBI unbinding from a sites (ttcCTCGGGaat) (SEQ ID NO:1), N=46), while lined bars represent BsoBI unbinding from P sites (aaaCTCGAGaga) (SEQ ID NO:2), N=12). Solid lines are predicted distributions based on the parameters shown in Table 1. All data are for a force loading rate of ˜60 pN/s. At this particular stretch rate, the two sites produce highly distinct unbinding signatures, as shown by both the data and the predicted PDF.

### DETAILED DESCRIPTION OF THE INVENTION

One aspect of the present invention is directed to a method of identifying the location of a binding site for a binding molecule on a double-stranded nucleic acid molecule. A double-stranded nucleic acid molecule having a first nucleic acid strand and a second nucleic acid strand is provided. In addition, a binding molecule is provided. The double-stranded nucleic acid molecule and the binding molecule are brought into contact under conditions effective to yield a modified nucleic acid molecule, including the binding molecule bound to the double-stranded nucleic acid molecule. The first and second nucleic acid strands are unzipped from one another under conditions effective to disrupt the binding molecule, if any, from being bound to the double-stranded nucleic acid molecule. Any binding site locations for a given binding molecule on the nucleic acid molecule is identified by comparing the force required to unzip the first and second nucleic acid strands from one another with the force required to unzip the nucleic acid molecule with no binding molecule bound thereto. Locations with a change in the force required to unzip the modified nucleic acid molecule compared to the force required to unzip the nucleic acid molecule with no binding molecule bound thereto are binding sites on the modified nucleic acid molecule.

As used herein, the terms “disrupt” and “disruption” are generally defined as meaning a force-induced change in the binding molecule/double-stranded nucleic acid complex (e.g., protein-DNA complex). Disruption is accompanied by unzipping of the nucleic acid molecule and an increase in length of single-stranded nucleic acid created. More specifically, but without limitation, disruption may include: (1) causing a binding molecule to completely release from a nucleic acid molecule; (2) causing the binding molecule to release from one strand only of a nucleic acid molecule, (3) causing the binding molecule to adopt a less tightly bound conformation with the nucleic acid molecule; (4) causing the binding molecule to separate into two or more parts, which may release from the nucleic acid molecule, or which may remain associated with the nucleic acid molecule.

Another aspect of the present invention is directed to a method of directly determining an equilibrium association constant of a target binding molecule specific to a double-stranded nucleic acid molecule. This involves providing a double-stranded nucleic acid molecule suspected of having a binding site for the target binding molecule, where the double-stranded nucleic acid molecule has both a first nucleic acid strand and a second nucleic acid strand. The target binding molecule is also provided. The double-stranded nucleic acid molecule and the target binding molecule are contacted with one another under conditions effective to yield a modified nucleic acid molecule. The modified nucleic acid molecule includes the target binding molecule bound to the double-stranded nucleic acid molecule. The first and second nucleic acid strands are unzipped from one another under conditions effective to disrupt the target binding molecule, if any, bound to the double-stranded nucleic acid molecule. A determination is then made as to whether a binding site on the double stranded nucleic acid molecule is occupied by the target binding molecule. This is done by comparing the force required to unzip the first and second nucleic acid strands from one another with the force required to unzip the double-stranded nucleic acid molecule having no target binding molecule bound to the double-stranded nucleic acid molecule. A change in force required to unzip the double-stranded nucleic acid molecule having no target binding molecule indicates the presence of a target binding molecule. The above steps (as described in this paragraph) are repeated at least one time, and the data is used to calculate the ratio of occupied to unoccupied binding sites as a result of carrying out the above steps of the method of the present invention. The resulting ratio is divided by the bulk protein concentrations of bound to unbound target binding molecules to the suspected binding site based on the determination described above relating to change of force required to unzip the double-stranded nucleic acid molecule. This ratio is the equilibrium constant of the target binding molecule to the suspected binding site.

Another aspect of the present invention is directed to a method of determining the dynamic force signature of a target binding molecule in relation to a binding site on a double-stranded nucleic acid molecule. This involves providing a double-stranded nucleic acid molecule suspected of having a binding site for the target binding molecule, where the double-stranded nucleic acid molecule has both a first nucleic acid strand and second nucleic acid strand. The double-stranded nucleic acid molecule and the target binding molecule are contacted with one another under conditions effective to yield a modified nucleic acid molecule having at least one binding complex (i.e., a complex including the target binding molecule bound to the double-stranded nucleic acid molecule). The first and second nucleic acid strands are unzipped from one another under conditions effective to disrupt the target binding molecule, if any, bound to the double-stranded nucleic acid molecule. A determination is made as to the unzipping disruption location, starting force, peak force for disruption, and force loading pattern for the target binding molecule. The previous steps of this paragraph are repeated, and, thereafter, the dynamic force signature for the binding molecule are calculated based on the unzipping location, the starting force, the peak force, and the force loading pattern determinations.

As used herein, the term “dynamic force signature” is defined as the behavior of a given binding molecule, e.g., protein/DNA complex—or more generally, molecule-molecule bound complexes—when subjected to unzipping forces. The behavior is characterized by analyzing the forces when the complex disrupts. If the disruption force is higher than with naked DNA unzipping, then the “peak force” is analyzed. If the disruption force is lower, then the “valley force” is analyzed.

The dynamic force signature of a given complex (for example, protein bound to DNA) is comprised of (A) the distribution of disruption forces observed under a given force loading pattern and (B) the change in the distribution from (A) as the force loading pattern is changed (for example, slower and faster loading rates).

The dynamic force signature for a given complex is determined by the underlying energetics of the interaction. Since different species of a molecule will usually have different underlying energetics, with sufficient resolution and quantity of data, distinguishable dynamic force signatures should be determinable. The general implications are two fold, as described below: First, distinct dynamic force signatures allow many kinds of assays where the nature of the binding complex can be determined from the dynamic force signature. For example, one could determine whether molecule (X) or molecule (Y) was bound when the nucleic acid was unzipped Another example includes the analysis of whether a third molecule or compound was present or acting on the binding complex. This can be analyzed by determining any modification in the dynamic force signature when the nucleic acid was unzipped, compared to the dynamic force signature of only the binding complex (i.e., in the absence of the third molecule or compound). Second, Dynamic Force Spectroscopy (DFS) (Evans, “Probing the Relation Between Force—Lifetime—and Chemistry in Single Molecular Bonds,” Ann. Rev. Biophys. Biomol. Struct. 30:105-28 (2001), the entire disclosure of which is hereby incorporated by reference in its entirety) can be applied to the dynamic force signatures. This allows one to obtain quantitative information about the energetics of the bound complex (i.e., the interaction of the protein with the DNA).

In addition, the method of the present invention for determining the dynamic force signature has many practical applications. There are two broad categories of these applications: (A) methods that rely on distinct signatures to determine what sort of complex was unzipped through can be used in tandem with the methods of the present invention; and (B) methods that rely on applying dynamic force spectroscopy (commonly known as DFS) to obtain quantitative measurements.

The first group (A) (i.e., methods that rely on distinct signatures to determine what sort of complex was unzipped through can be used in tandem with the methods of the present invention) includes the following types of studies: Analyzing the competition between two or more binding proteins can be done. For example, one could include two binding proteins during the binding and unzipping process (and at the same time) and use the dynamic signature to see which of the two proteins binds at a specific site. This allows for the direct measurement of relative affinities. Dynamic signatures can also be used to determine the nature of an unknown binding site on a DNA molecule. For example, one can determine whether the site is CTCGGG or CTCGAG. Dynamic signatures can further be used to determine the nature and/or mixture of an unknown binding mixture. For example, the dynamic signature may depend on phosphorylation, acetylation, or methylation state, and many other variations in a binding protein. The dynamic signature can further be used to monitor the binding of a third party molecule to the known complex. For example, a base complex is a protein-DNA complex for which we know the signature. By measuring a change in this signature, one can see whether a third protein is interacting with the binding protein. By varying concentration of the third-party protein one can indirectly measure the binding affinity for the protein-protein interaction, using the dynamic signature as a reporter for the interaction.

The second group (A) (i.e., methods that rely on applying dynamic force spectroscopy (commonly known as DFS) to obtain quantitative measurements) includes the following types of studies: Quantitative information about the energy landscape of the binding complex can be obtained. This information can then be used to draw conclusions about binding rate constants (specifically “off-rate”). Further, one can observe how this information changes when the system is tweaked and then draw conclusions from any changes or lack of changes. Examples of different tweaking events include changing the buffer conditions (e.g., ionic strength, water activity), adding third-party molecules (e.g., other proteins; drugs), changing temperatures, and slightly modifying the binding protein. More specifically, the method of the present invention can be used for determining whether the target binding molecule is a drug candidate which either binds to the double-stranded nucleic acid molecule or inhibits binding of another material to the double-stranded nucleic acid molecule. Further, quantitative numbers from two different species can be compared to draw conclusions. For example, mutating a single amino acid of a protein may raise or lower the energy of a certain part of the energy landscape. The dynamic signature may be used to measure a change (or lack thereof) in this energy.

Another aspect of the present invention is directed to a method of identifying whether a target nucleic acid molecule is present in a sample. This involves providing a sample potentially containing a double-stranded target nucleic acid molecule having a first nucleic acid strand and a second nucleic acid strand. A protein which binds to a binding site on the target nucleic acid molecule is provided. The sample and the protein are contacted with one another under conditions effective to permit the protein to bind to the target nucleic acid molecule, if present in the sample. The first and second nucleic acid strands are unzipped from one another under conditions effective to separate the two strands. Thereafter, the presence of any target nucleic acid molecules is identified by measuring the force required to unzip the first and second nucleic acid strands from one another, where a change in force indicates the presence of the target nucleic acid molecule in the sample.

An additional aspect of the present invention is directed to a method of producing a restriction map for a nucleic acid molecule. This involves providing a double-stranded nucleic acid molecule having a first nucleic acid strand and a second nucleic acid strand. A restriction endonuclease is also provided. The double-stranded nucleic acid molecule and the restriction endonuclease are contacted with one another under conditions effective to yield a binding complex of the restriction endonuclease bound to the double-stranded nucleic acid molecule, while prohibiting the restriction endonuclease from cutting the nucleic acid molecule. The first and second nucleic acid strands are unzipped from one another under conditions effective to separate the first and second nucleic acid strands from one another. Thereafter, the binding site locations are identified, if any, for the restriction endonuclease on the nucleic acid molecule. This is done by comparing the force required to unzip the first and second nucleic acid strands from one another with the force required to unzip the double-stranded nucleic acid molecule having no restriction endonuclease bound thereto, where a change in force required to unzip the double-stranded nucleic acid molecule having no restriction endonuclease indicates the location of a binding site for the restriction endonuclease on the double-stranded nucleic acid molecule. This method can be repeated for each additional restriction endonuclease to be mapped. Various suitable restriction endonucleases can be used in the methods of the present invention, including, without limitation, EcoRI, BsoBI, XhoI, HindIII, NotI, BamHI, HaeIII, HpaI, PstI, Sau3A, SamI, SstI, XmaI, and combinations thereof.

Yet another aspect of the present invention is directed to a method of identifying whether a target protein is present in a sample. This involves providing a sample potentially containing a target protein and providing a double-stranded nucleic acid molecule. The double-stranded nucleic acid molecule has a first nucleic acid strand and a second nucleic acid strand, as well as a binding site to which the target protein binds. The sample and the double-stranded nucleic acid molecule are contacted with one another under conditions effective to permit the target protein, if present, to bind to the double-stranded nucleic acid molecule. The first and second nucleic acid strands are unzipped under conditions effective to separate the first and second nucleic acid strands from one another. Thereafter, the presence of the target protein is identified by comparing the force required to unzip the first and second nucleic acid strands from one another with the force required to unzip the double-stranded nucleic acid molecule having no target protein bound thereto, where a change in force required to unzip the double-stranded nucleic acid molecule having no target protein indicates the presence the target protein in the sample.

As used herein, the term “unzipping” generally means the separation of the two strands of a double-stranded nucleic acid molecule. In one embodiment, this unzipping is achieved by identifying a location on the double-stranded nucleic acid to begin the unzipping process. This location may be described as the unzipping initiation site. This unzipping initiation site can be used to determine the beginning point of unzipping of the two strands of the double-stranded nucleic acid molecule. Further, the unzipping initiation site determines where the 5′- and 3′-ends of the two strands of the double-stranded nucleic acid are.

In one aspect of the present invention, prior to unzipping, double-stranded nucleic acid may be secured (i.e., anchored) to a securing component. Suitable securing components for use in the methods of the present invention include, without limitation, a microwell, a microtiter plate, a microscope slide (Koch et al., “Probing Protein-DNA Interactions by Unzipping a Single DNA Double Helix,” Biophys. J. 83:1098-1105 (2002); Brower-Toland et al., “Mechanical Disruption of Individual Nucleosomes Reveals a Reversible Multistage Release of DNA,” Proc. Nat'l Acad. Sci. 99:1960-1965 (2002); Bockelmann et al., “Molecular Stick-Slip Motion Revealed by Opening DNA with Piconewton Forces,” Physical Review Letters 79(22):4489-4492 (1997), the entire disclosures of which are hereby incorporated by reference in their entirety), a miscroscope coverslip (Koch et al., “Probing Protein-DNA Interactions by Unzipping a Single DNA Double Helix,” Biophys. J. 83:1098-1105 (2002); Brower-Toland et al., “Mechanical Disruption of Individual Nucleosomes Reveals a Reversible Multistage Release of DNA,” Proc. Nat'l Acad. Sci. 99:1960-1965 (2002); Bockelmann et al., “Molecular Stick-Slip Motion Revealed by Opening DNA with Piconewton Forces,” Physical Review Letters 79(22):4489-4492 (1997), the entire disclosures of which are hereby incorporated by reference in their entirety), a microsphere (Koch et al., “Probing Protein-DNA Interactions by Unzipping a Single DNA Double Helix,” Biophys. J. 83:1098-1105 (2002); Brower-Toland et al., “Mechanical Disruption of Individual Nucleosomes Reveals a Reversible Multistage Release of DNA,” Proc. Nat'l Acad. Sci. 99:1960-1965 (2002); Bockelmann et al., “Molecular Stick-Slip Motion Revealed by Opening DNA with Piconewton Forces,” Physical Review Letters 79(22):4489-4492 (1997), the entire disclosures of which are hereby incorporated by reference in their entirety), a column, a disc, a membrane, a film, a micropipette (Essevaz-Roulet et al., “Mechanical Separation of the Complementary Strands of DNA,” Proc. Natl. Acad. Sci. USA 94:11935-11940 (1997), the entire disclosure of which is hereby incorporated by reference in its entirety), a viscous drag force, a nanotube (Umemura et al., “Atomic Force Microscopy of RecA-DNA Complexes Using a Carbon Nanotube Tip, Biochemical and Biophysical Research Communications,” 281:390-395 (2001), the entire disclosure of which is hereby incorporated by reference in its entirety), a tip of an optical fiber (Cluzel et al., “DNA: An Extensible Molecule,” Science 271(5250):792-4 (1996), the entire disclosure of which is hereby incorporated by reference in its entirety), and a tip of a scanning probe (Clausen-Schaumann et al., “Mechanical Stability of Single DNA Molecules,” Biophys. J. 78:1997-2007 (1997), the entire disclosure of which is hereby incorporated by reference in its entirety). In one embodiment, the methods of the present invention may involve securing a 5′-portion of the first nucleic acid strand to a first securing component prior to said unzipping and securing a 3′-portion of the second nucleic acid strand to a second securing component prior to said unzipping. This securing can be achieved using a nucleic acid bonding technique. Suitable nucleic acid bonding techniques include, without limitation, streptavidin/biotin binding and antibody/antigen binding. However, many other different types of nucleic acid bonding techniques well known in the art can be used in accordance with the methods of the present invention.

In one embodiment of the present invention, securing components can be used to secure the ends of the two strands of the double-stranded nucleic acid molecule around the unzipping initiation site. Thus, one of the nucleic acid strands (e.g., the first nucleic acid strand) can be secured, directly or indirectly, by a first securing component, while the other nucleic acid strand (e.g., the second nucleic acid strand) can be secured, directly or indirectly, by a second securing component. The use of the first and second securing components can be assist in unzipping the nucleic acid strands by using such unzipping techniques as: (1) moving the first securing component away from the second securing component, where the second securing component is kept relatively stationary; (2) moving the second securing component away from the first securing component, where the second securing component is kept relatively stationary; or (3) simultaneously moving the first and second securing components away from one another. Suitable methods of keeping the first or second securing components relatively stationary includes, without limitation, using a technique such as optical trapping technology (Koch et al., “Probing Protein-DNA Interactions by Unzipping a Single DNA Double Helix,” Biophys. J. 83:1098-1105 (2002); Brower-Toland et al., “Mechanical Disruption of Individual Nucleosomes Reveals a Reversible Multistage Release of DNA,” Proc. Nat'l Acad. Sci. 99:1960-1965 (2002); Bockelmann et al., “Molecular Stick-Slip Motion Revealed by Opening DNA with Piconewton Forces,” Physical Review Letters 79(22):4489-4492 (1997), the entire disclosures of which are hereby incorporated by reference in their entirety), micropipette technology (Essevaz-Roulet et al., “Mechanical Separation of the Complementary Strands of DNA,” Proc. Natl. Acad. Sci. USA 94:11935-11940 (1997), the entire disclosure of which is hereby incorporated by reference in its entirety), atomic force microscopy (Clausen-Schaumann et al., “Mechanical Stability of Single DNA Molecules,” Biophys. J. 78:1997-2007 (1997), the entire disclosure of which is hereby incorporated by reference in its entirety), magnetic force microscopy (Strick et al., “The Elasticity of a Single Supercoiled DNA Molecule,” Science 271:1835-1837 (1996), the entire disclosure of which is hereby incorporated by reference in its entirety), optical fiber force transducer technology (Cluzel et al., “DNA: An Extensible Molecule,” Science 271(5250):792-4 (1996), the entire disclosure of which is hereby incorporated by reference in its entirety), nano-fabricated cantilever or tip technology, micro-fabricated cantilever or tip technology (Mabry et al., “Developments for Inverted Atomic Force Microscopy,” Ultramicroscopy 91(1-4):73-82 (2002), the entire disclosure of which is hereby incorporated by reference in its entirety), nanotube technology (Umemura et al., “Atomic Force Microscopy of RecA-DNA Complexes Using a Carbon Nanotube Tip, Biochemical and Biophysical Research Communications,” 281:390-395 (2001), the entire disclosure of which is hereby incorporated by reference in its entirety), and microelectromechanical technology (Lin et al., “Miniature Heart Cell Force Transducer System Implemented in MEMS Technology,” IEEE Trans. Biomed. Eng. 48(9):996-1006 (2001), the entire disclosure of which is hereby incorporated by reference in its entirety).

As describe herein, the change in force is either an increase or decrease in the force required to unzip the first and second nucleic acid strands of the modified nucleic acid molecule compared to the force required to unzip the control nucleic acid molecule with no binding molecule bound thereto. Various methods of measuring force can be used.

In one embodiment, the binding sites for the binding molecule on a double-stranded nucleic acid molecule can be identified by measuring force using force sensor technology. Suitable types of force sensor technology include, but are not limited to, optical trapping technology (Koch et al., “Probing Protein-DNA Interactions by Unzipping a Single DNA Double Helix,” Biophys. J. 83:1098-1105 (2002); Brower-Toland et al., “Mechanical Disruption of Individual Nucleosomes Reveals a Reversible Multistage Release of DNA,” Proc. Nat'l Acad. Sci. 99:1960-1965 (2002); Bockelmann et al., “Molecular Stick-Slip Motion Revealed by Opening DNA with Piconewton Forces,” Physical Review Letters 79(22):4489-4492 (1997), the entire disclosures of which are hereby incorporated by reference in their entirety), micropipette technology (Essevaz-Roulet et al., “Mechanical Separation of the Complementary Strands of DNA,” Proc. Natl. Acad. Sci. USA 94:11935-11940 (1997), the entire disclosure of which is hereby incorporated by reference in its entirety), atomic force microscopy (Clausen-Schaumann et al., “Mechanical Stability of Single DNA Molecules,” Biophys. J. 78:1997-2007 (1997), the entire disclosure of which is hereby incorporated by reference in its entirety), magnetic force microscopy (Strick et al., “The Elasticity of a Single Supercoiled DNA Molecule,” Science 271:1835-1837 (1996), the entire disclosure of which is hereby incorporated by reference in its entirety), optical fiber force transducer technology (Cluzel et al., “DNA: An Extensible Molecule,” Science 271(5250):792-4 (1996), the entire disclosure of which is hereby incorporated by reference in its entirety), nano-fabricated cantilever or tip technology, micro-fabricated cantilever or tip technology (Mabry et al., “Developments for Inverted Atomic Force Microscopy,” Ultramicroscopy 91(1-4):73-82 (2002), the entire disclosure of which is hereby incorporated by reference in its entirety), nanotube technology (Umemura et al., “Atomic Force Microscopy of RecA-DNA Complexes Using a Carbon Nanotube Tip, Biochemical and Biophysical Research Communications,” 281:390-395 (2001), the entire disclosure of which is hereby incorporated by reference in its entirety), and microelectromechanical technology (Lin et al., “Miniature Heart Cell Force Transducer System Implemented in MEMS Technology,” IEEE Trans. Biomed. Eng. 48(9):996-1006 (2001), the entire disclosure of which is hereby incorporated by reference in its entirety). More specifically, the optical trapping technology may include a feedback-enhanced optical trap.

As referenced in the various methods of the present invention, the various types of binding molecules may include, without limitation, polypeptides, an oligonucleotide, inorganic chemical compounds, organic chemical compounds, and PNAs. More specifically, the polypeptide may be a restriction endonuclease, a polymerase, a helicase, a nuclease, an isomerase, a ligase, activators, repressors, and a histone.

Also as reference herein with respect to the methods of the present invention, the double-stranded nucleic acid molecule may be a double-stranded DNA molecule, a double-stranded RNA molecule, or a DNA/RNA duplex molecule.

The UFAPA technique can be used to study protein-DNA interactions. For example, the restriction endonucleases are a well-studied class of DNA-binding proteins. EcoRI and other restriction endonucleases have been important tools in the development of modem molecular biology, and have also served as useful models for other protein-DNA interactions. UFAPA has been used to detect EcoRI binding at two canonical sites separated by 11 bp. It has also been used for non-catalytic restriction mapping of DNA using various restriction endonucleases (e.g., BsoBI, XhoI, and EcoRI). UFAPA has further been used to determine the cation concentration dependence of the equilibrium association constant of EcoRI binding.

In one embodiment of the methods of the present invention, optical trapping instrumentation is used. For example, the UFAPA technique works well with optical trapping instrumentation. The accuracy and precision of UFAPA using optical trapping nanometry can be further enhanced by instrumentation improvements.

The trapping setup can be equipped with an axial position sensor to eliminate uncertainty in the trap height estimate relative to the microscope coverslip. This will improve the extension determination, especially when the tether length is short.

The trapping setup can be equipped with a temperature controlled sample stage so that the temperature can be maintained within a fraction of a degree. A change in temperature will produced a change in the force-extension relation used to convert to unzipping index j, as well as in the measurements of equilibrium association constants and dynamic signatures.

In order to increase the force range above the B-S transition (˜65 pN), the dsDNA segment can be removed. In conjunction with removal of the dsDNA segment, covalent linkages between DNA and surfaces would increase the force range with optical tweezers. ssDNA has been shown to withstand several hundred pN.

Alternatively, a system that winds the two single strands around a “spool,” thus keeping the length of ssDNA fixed, short, and taught, would be advantageous for use with the methods of the present invention. In principle, this would allow mapping an entire chromosome with optimal resolution.

In addition to using optical trapping techniques, other instrumentation can be used alongside the methods of the present invention.

Unzipping DNA in UFAPA can be achieved using a number of other single molecule approaches besides the optical trapping nanometry technique. For example, unzipping can be achieved with atomic force microscopy (AFM), micropipette, magnetic force microscope, and so on.

Unzipping may also be achieved using microelectromechanical (MEM) technology. Micro-mechanical force transducers can be used to unzip the DNA double helix. Micro-mechanical sensors can be used to detect forces and displacements.

The methods of the present invention may also be automated, as described below. Microfluidics could be used to aid in rapid screening of different molecules. To streamline the process of detection, microfabricated channels could be used to deliver different DNA binding proteins to the sample, or a sample chamber could be fabricated with multiple wells, each of which contains a sample with a different DNA molecule or protein. For mapping genomes, it is possible that Bacterial Artificial Chromosomes (BAC) libraries would lend themselves to our current implementation of the methods of the present invention, including, for example, the UFAPA technique. Multiple cloning sites of BACs could be used for ligating an unknown sequence onto our dig-/bio-anchoring segment. Also, genomic DNA (as opposed to a library such as BACs) could be shotgun UFAPA mapped by digesting with, for example, NotI, followed by mapping of random fragments which have been ligated to the anchoring segment via the NotI sticky end.

### EXAMPLES

The Examples set forth below are for illustrative purposes only and are not intended to limit, in any way, the scope of the present invention.

### Example 1

### Probing Protein-DNA Interactions by Unzipping a Single DNA Double Helix

A single DNA double helix was unzipped in the presence of DNA-binding proteins using a feedback-enhanced optical trap. When the unzipping fork in a DNA reached a bound protein molecule, a dramatic increase in the tension in the DNA was observed, followed by a sudden tension reduction. Analysis of the unzipping force throughout an unbinding “event” revealed information about the spatial location and dynamic nature of the protein-DNA complex. The capacity of UFAPA to spatially locate protein-DNA interactions is demonstrated by non-catalytic restriction mapping on a 4 kb DNA with three restriction enzymes (BsoBI, XhoI, and EcoRI). A restriction map for a given restriction enzyme was generated with an accuracy of about 25 bp. UFAPA also allows direct determination of the site-specific equilibrium association constant (K_{A}) for a DNA-binding protein. This capability is demonstrated by measuring the cation concentration dependence of K_{A} for EcoRI binding. The measured values are in good agreement with previous measurements of K_{A} over an intermediate range of cation concentration. The theory of dynamic force spectroscopy (DFS) also can be incorporated into UFAPA. The probability distribution functions (PDFs) for unbinding events was observed to obey the simple model presented by DFS. Examination of the three binding species showed that the different underlying binding energetics produce different DFS signatures and corresponding characteristic bond forces, f_{β}, and force free bond lifetimes, t_{off}. These results demonstrate the potential utility of UFAPA for future studies of site-specific protein-DNA interactions.

### Example 2

### Summary of the Experimental Configuration Relating to Probing Protein-DNA Interactions by Unzipping a Single DNA Double Helix

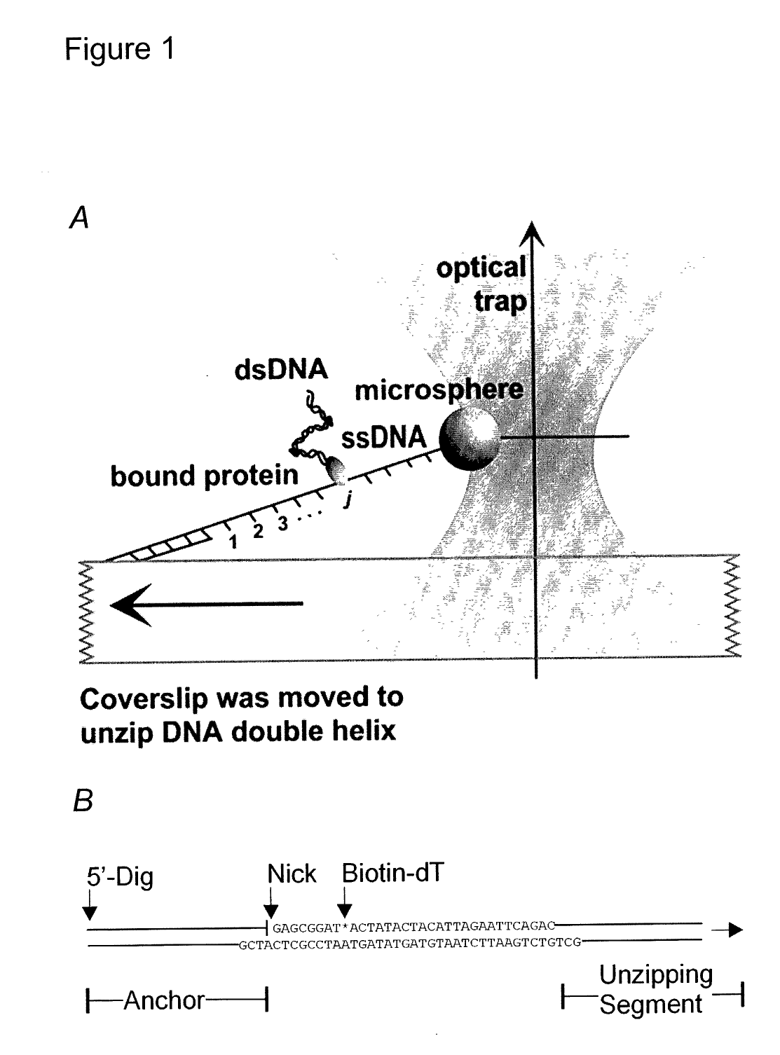

The experimental configuration is shown in FIG. 1A. One strand of a double-stranded (ds) DNA molecule to be unzipped was attached to the surface of a microscope coverslip while the other strand, which originated from the same end, was attached to a polystyrene microsphere. To unzip the dsDNA, the two single strands of the DNA molecule were pulled apart by moving the coverslip, while holding the microsphere in a fixed position with a feedback-enhanced optical trap. The number of unzipped base pairs is referenced by an unzipping index j. This configuration is a combination of those used by others in the field (Bockelmann et al., “Molecular Stick-Slip Motion Revealed by Opening DNA with Piconewton Forces,” Phys. Rev. Lett. 79:4489-4492 (1997); Bockelman et al., “DNA Strand Separation Studied by Single Molecule Force Measurements,” Phys. Rev. E. 58:2386-2394 (1998); Essevaz-Roulet et al., “Mechanical Separation of the Complementary Strands of DNA,” Proc. Nat'l Acad. Sci. USA 94:11935-11940 (1997); and Wang et al., “Force and Velocity Measured for Single Molecules of RNA Polymerase,” Science 282:902-907 (1998), the entire disclosures of which are incorporated by reference herein in their entirety).

### Example 3

### Biochemical Materials

DNA Molecules. The DNA molecule used for unzipping, was adapted from Bockelmann et al., “Molecular Stick-Slip Motion Revealed by Opening DNA with Piconewton Forces,” Phys. Rev. Lett. 79:4489-4492 (1997); Bockelman et al., “DNA Strand Separation Studied by Single Molecule Force Measurements,” Phys. Rev. E. 58:2386-2394 (1998); Essevaz-Roulet et al., “Mechanical Separation of the Complementary Strands of DNA,” Proc. Nat'l Acad. Sci. USA 94:11935-11940 (1997), the entire disclosures of which are hereby incorporated by reference in their entirety, and is shown in FIG. 1B. One end of the DNA was labeled with a digoxigenin (dig) for attachment to a coverslip via anti-digoxigenin (Roche Molecular Biochemicals, Indianapolis, Ind.). The nicked strand, 1.1 kb distant from the dig labeled end, was labeled with a biotin at 8 bp away from the nick for attachment to a streptavidin-coated 0.48 μm diameter microsphere (Bangs Laboratories, Inc., Fishers, Ind.). Therefore, when the DNA was unzipped by j bases, there were Nss=2j-8 bases in the ssDNA. The two insert oligos whose complete sequences are shown in FIG. 1B allowed for coupling the dig-labeled anchoring segment to the unzipping segment, via a 3′ overhang and a 5′ overhang on the bottom strand of the duplex.

The anchoring double-stranded segment (1120 bp) was derived from the rpoB gene contained in pRL574. The dig label was the result of PCR with a dig-labeled primer. After PCR, the segment was digested with BstXI (NEB), gel extracted, and ligated to the ATCG-3′ overhang of the insert duplex.

The repetitive unzipping segment used in most of the experiments was derived from pCP681 consisting of a sequence of 17 head-to-tail segments of the form xxxxyzxxxxyzxxxxy, where “x,” “y,” and “z” each represent nucleic acid sequences approximately 200 bps long, each of which were derived from 5S rRNA genes (see Logie et al., “Catalytic Activity of the Yeast SWI/SNF Complex on Reconstituted Nucleosome Arrays,” EMBO J. 16:6772-6782 (1997), the entire disclosure of which is hereby incorporated by reference in its entirety, for the corresponding 11 head-to-tail segments of the form xxxxyzxxxxy). pCP681 was digested with Earl (NEB), the 4.1 kb fragment was gel extracted, and then ligated to the 5′-GCT overhang of the insert duplex. For further EcoRI studies, a different unzipping segment was ligated via the same 5′-GCT overhang. This unzipping segment was the large fragment of an Earl digest of pBR322, resulting in a single EcoRI site ˜2.4 kb downstream from the nick. Unzipping constructs were attached to the dig surface by incubating at DNA concentrations ≦30 pM. Given a maximum DNA tethering efficiency of 10%, this is equivalent to a solution concentration of ≦3 pM.

Unzipping Buffer Conditions. Experiments with BsoBI and XhoI were performed at room temperature (23° C.) in a buffer containing 50 mM sodium phosphate buffer pH 7.0, 50 mM NaCl, 0.02% Tween-20, 10 mM EDTA. To facilitate better comparisons of future EcoRI results with previously reported results, mapping and equilibrium constants studies of EcoRI were performed at room temperature (23° C.) in a buffer containing 10 mM HEPES, pH 7.6, 1 mM EDTA, 50 μM DTT, 100 μg/ml BSA, 500 μg/ml Blotting Grade Blocker (Bio-Rad) and NaCl added to produce total Na+concentrations of 106 to 262 mM. All buffers did not contain Mg2+, which is required for catalytic activity of the restriction endonucleases. The Hepes buffer is similar to the buffer used by Ha et al., “Role of the Hydrophobic Effect in Stability of Site-Specific Protein-DNA Complexes,” J. Mol. Biol. 209:801-816 (1989), the entire disclosure of which is hereby incorporated by reference in its entirety, for temperature dependence studies of EcoRI binding to its site on pBR322. Because of the tendency for EcoRI to aggregate at lower ionic strengths (Jen-Jacobson et al., “Coordinate Ion Pair Formation Between EcoRI Endonuclease and DNA,” J. Biol. Chem. 258:14638-14646 (1983), the entire disclosure of which is hereby incorporated by reference in its entirety), the EcoRI mapping data were taken at 131 mM total Na+ concentration.

Enzymes. All enzymes were commercial grade, purchased from New England BioLabs (NEB) (Beverly, Mass.), and used without further purification.

To determine the molar concentration of actively binding EcoRI, an agarose gel mobility shift assay was performed. Various concentrations of EcoRI were incubated for 1 hour at room temperature with 10 nM of a 33 bp synthesized DNA duplex containing a single EcoRI binding site in 10 mM HEPES, pH 7.6, 1 mM EDTA, 50 μM DTT, 100 μg/ml BSA, 156-194 mM total Na+ concentration during incubation. These samples were then run on a 2.4% agarose gel at 4° C. to determine the fraction of DNA bound. Using this assay, the concentration of actively binding EcoRI molecules in the undiluted stock was determined to be 300 nM. This is about 40% of the expected 800 nM based on NEB's reported activity for this lot (62 kD dimer; 2×10⁶ U/mg specific activity; 100,000 U/ml stock concentration). The difference between the measured activity and that reported by NEB may reflect degradation of enzymatic activity or errors in NEB's reported unit concentration and specific activity. EcoRI equilibrium constant measurements were performed with NEB enzyme at concentrations from 50 to 6000 pM with the actual concentration chosen for maximum expected counting precision (See Results).

The concentrations of BsoBI and XhoI were determined from the company's reported unit concentration and specific activity of each enzyme—the actual active binding fraction was not determined. BsoBI and XhoI were used at respective concentrations of 0.7 nM (72 kD dimer; 4×10⁶ U/mg specific activity; 200 U/ml working concentration) and 4.6 nM (52 kD dimer; 1.7×10⁶ U/mg specific activity; 400 U/ml working concentration).

### Example 4

### Instrumentation and Calibration

The measurements were obtained using a single-beam optical trapping microscope. After passing through a single-mode optical fiber (Oz Optics, Carp, ON) and an acousto-optic deflector (NEOS Technologies, Inc., Melbourne, Fla.), 1064 nm laser light (Spectra-Physics Lasers, Inc. Mountain View, Calif.) was focused onto the sample plane using a 100×, 1.4 NA, oil immersion objective on an Eclipse TE200 DIC microscope (Nikon USA, Melville, N.Y.). After interacting with a trapped microsphere, the laser light was collected by a 1.4 NA oil immersion condenser and projected onto a quadrant photodiode (Hamamatsu, Bridgewater, N.J.). The photocurrents from each quadrant of the photodiode were amplified and converted to voltage signals using a position detection amplifier (On-Trak Photonics, Inc., Lake Forest, California). The position of the optical trap relative to the sample was adjusted with a servo-controlled 1-D piezoelectric stage (Physik Instrumente GmbH & Co, Waldbronn, Germany). Analog voltage signals from the position detector and stage position sensor were anti-alias filtered at 5 kHz (Krohn-Hite, Avon, Mass.) and digitized at 7 to 13 kHz for each channel using a multiplexed analog to digital conversion PCI board (National Instruments Corporation, Austin, Tex.).

The calibration and data conversion methods of the instrument were adapted from those used by Wang et al. (Wang et al., “Force and Velocity Measured for Single Molecules of RNA Polymerase,” Science 282:902-907 (1998); Wang et al., “Stretching DNA with Optical Tweezers,” Biophys. J. 72:1335-1346 (1997), the entire disclosures of which are hereby incorporated by reference in their entirety). In brief, the first step of the calibration determined the position of the trap center relative to the beam waist and the height of the trap center relative to the coverslip. The second step of the calibration determined the position detector sensitivity and trap stiffness. The third step of the calibration located the anchor position of the DNA on the coverslip, and was performed prior to each unzipping measurement by stretching a DNA at low load (<5 pN, not sufficient to unzip). These calibrations were subsequently used to convert data into force and extension for an actual unzipping experiment.

### Example 5

### Determination of the Force-Extension Relations

Elastic parameters of both dsDNA and single-stranded(ss) DNA are necessary for the interpretation of the data. The elastic parameters of dsDNA were obtained from Wang et al., “Stretching DNA with Optical Tweezers,” Biophys. J. 72:1335-1346 (1997), the entire disclosure of which is hereby incorporated by reference in its entirety, which used an extensible worm-like-chain model (Marko et al., “Stretching DNA,” Macromolecules 28:7016-7018 (1995), the entire disclosure of which is hereby incorporated by reference in its entirety): the contour length per base 0.338 nm, the persistence length of DNA 43.1 nm, and the stretch modulus 1205 pN. To obtain the elastic parameters of ssDNA, a modified version of the DNA molecule was constructed that included a capped end on the double-stranded part that was to be unzipped (Bockelmann et al., “Molecular Stick-Slip Motion Revealed by Opening DNA with Piconewton Forces,” Phys. Rev. Lett. 79:4489-4492 (1997), the entire disclosure of which is hereby incorporated by reference in its entirety). First, this DNA was completely unzipped (forces 12-17 pN). This resulted in a rather extended molecule with dsDNA (at the coverslip anchor) and ssDNA in series. This unzipped DNA was then stretched to a higher force up to 50 pN to obtain the force-extension curve, which reflects elastic contributions from both the dsDNA and ssDNA. Given the elastic parameters of dsDNA, this curve allowed the determination of the elastic properties of ssDNA using an extensible freely-jointed-chain model (Smith et al., “Overstretching B-DNA: The Elastic Response of Individual Double-Stranded and Single-Stranded DNA Molecules,” Science 271:795-799 (1996), the entire disclosure of which is hereby incorporated by reference in its entirety): a contour length per base of 0.539 nm, a persistence length of 0.796 nm, and a stretch modulus of 580 pN.

### Example 6

### Unzipping Data Acquisition

To unzip a DNA double helix as shown in FIG. 1A, the coverslip was moved relative to the trapped microsphere with a piezoelectric stage to stretch the DNA under either a velocity clamp, a proportional velocity clamp, or a loading rate clamp. Both of these clamps were implemented with digital feedback, with an average rate for a complete feedback cycle of 7-13 kHz. In the velocity clamp mode, the coverslip was moved at a constant velocity νₛ (in nm/s) relative to the trapped microsphere, whose position was kept constant by modulating the light intensity (trap stiffness) of the trapping laser. Unzipping, during which dsDNA was converted to ssDNA, was observed as a reduction in the tension of the DNA. In the proportional velocity clamp mode, the coverslip was moved at a velocity νₛ that was proportional to the number of unzipped bases, Nss, calculated at real time, while the position of the microsphere was kept constant by modulating the light intensity (trap stiffness) of the trapping laser. In other words, in the proportional velocity clamp mode

$$\frac{{v}_{s}}{{N}_{s s}} ,$$

rather than νₛ was held constant. Unzipping was observed as a reduction in the tension of the DNA and a corresponding increase in the velocity of stretching. In the loading rate clamp,

$$f \frac{{v}_{s}}{{N}_{s s}}$$

was held constant, resulting in linear force loading rates for ssDNA stretching. The velocity clamp is rather straightforward as a method of stretching and was used in some of the experiments, whereas the proportional velocity clamp is an enhancement to account for the increasing compliance of the ssDNA as the construct is unzipped. The loading rate clamp is a further enhancement to account for increasing compliance from the lengthening of ssDNA while also accounting for decreasing compliance from the increase in force. The proportional velocity clamp and loading rate claimp allow UFAPA studies to quantitatively analyze the forces of unbinding events at different locations on the DNA.

A number of experiments were carried out to demonstrate the capability of the UFAPA approach to locate DNA-binding sites, and to assess the dynamic signatures of protein-DNA interactions. The DNA-binding proteins used here were restriction enzymes (BsoBI, XhoI, and EcoRI). As shown in FIG. 1A, tethered DNA was incubated with a restriction enzyme in the absence of Mg²⁺ which allowed the restriction enzyme to bind to its cognate site without cutting the DNA molecule. Before unzipping, the DNA and protein were incubated together for 15 minutes to allow them to come to equilibrium. Longer incubation times did not increase the fraction of detectable bound complexes.

### Example 7

### Detection of Bound Proteins

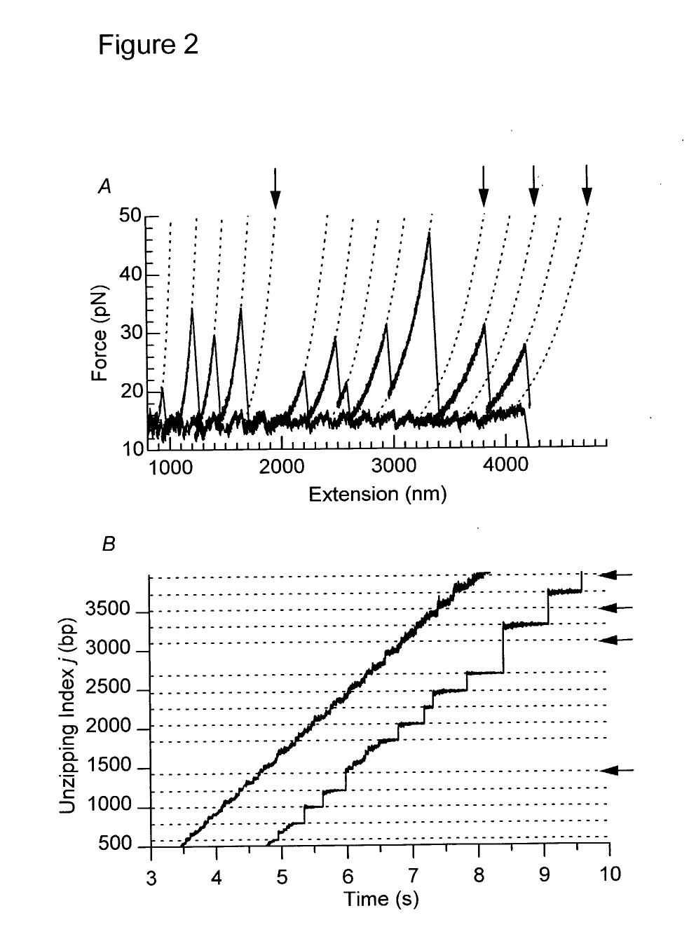

As the DNA was unzipped, the tension (force) and extension of the DNA were monitored continuously. An example of data is shown in FIG. 2A, which is a plot of the force-extension relation for an unzipping process that used a velocity clamp at 700 nm/s. The force-extension curve in the presence of BsoBI (red curve) differs dramatically from that of naked DNA (black curve). The unzipping force for naked DNA was rather uniform (12-17 pN), whereas unzipping in the presence of BsoBI produced a series of dramatic increases in force (up to 40 pN) with each increase followed by a rapid relaxation. The high force events observed for unzipping of DNA in the presence of BsoBI rise from a baseline which corresponds to the force curve obtained from unzipping of naked DNA. These high-force events presumably correspond to the resistance of BsoBI to unzipping and its subsequent unbinding from the DNA double helix.

In order to determine where a protein binds, the unzipping index j (see FIG. 1) must be converted from force-extension curves. This conversion relies on the elastic parameters of the stretched DNA (which was composed of both ssDNA and dsDNA). It uses a method similar to that used by Wang et al., “Force and Velocity Measured for Single Molecules of RNA Polymerase,” Science 282:902-907 (1998), the entire disclosure of which is hereby incorporated by reference in its entirety, to compute the DNA tether length during a single molecule transcription experiment. The converted data from FIG. 2A are shown in FIG. 2B, where j is plotted as a function of time. Compared with the naked DNA curve, the BsoBI curve shows a pronounced staircase pattern at each protein disruption event, due to clamping of the helix by the bound BsoBI. The locations of the plateaus clearly indicate the locations of the BsoBI binding sites on the DNA sequence. These measured binding sites agree well with the expected sites, which are indicated by the dotted horizontal lines in the plot.

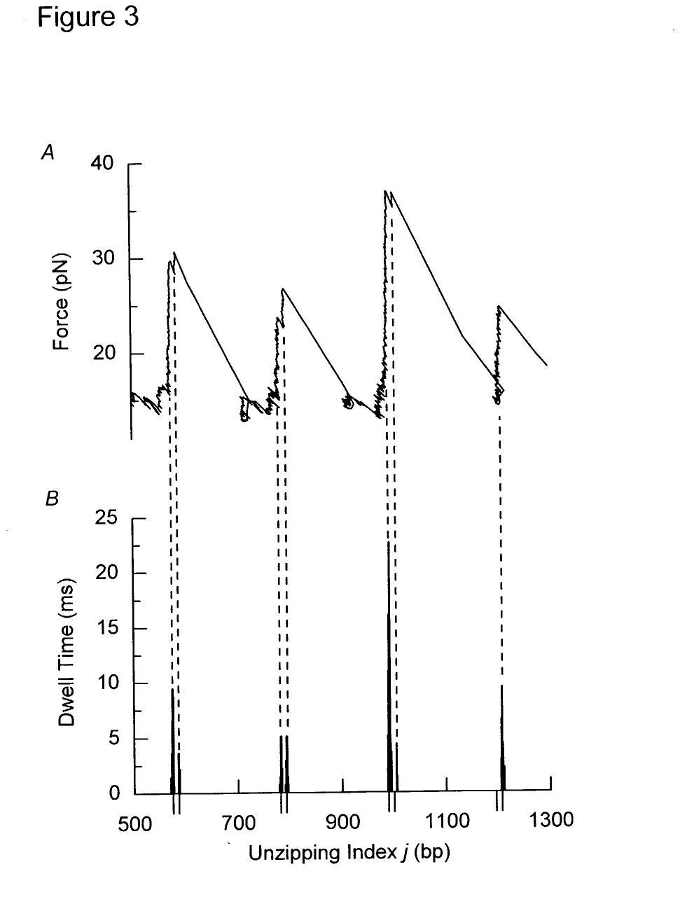

FIG. 3 illustrates the high resolution of the unzipping technique for ascertaining the location of one bound protein relative to another. FIG. 3A shows force versus unzipping index j for unzipping carried out in the presence of EcoRI using a velocity clamp at 280 nm/s. The DNA molecule contains two expected closely spaced EcoRI sites (vertical bars under the horizontal axis) differing by only 11 bp within each repeat of the tandem repeat sequence. Bound EcoRI was detected by a sudden rise in the force for unzipping. When EcoRI binding to one of these sites was disrupted, the DNA double helix unzipped and the tension dropped until it reached the level characteristic of that for unzipping naked DNA or until another bound EcoRI was encountered by the unzipping fork. As demonstrated by the doublet peaks around j=600, 800, and 1000 bp in FIG. 3A, binding sites that differ by as little as 11 bp can be readily resolved. To facilitate location of binding sites, a plot of dwell time versus unzipping index j is shown in FIG. 3B. Only data corresponding to forces >20 pN are included in this plot and the bin size for unzipping index is 1 bp. The standard deviation of a peak is 3 bp, the resolution limit for the determination of the location of one bound protein relative to another.

### Example 8

### Mapping of Bound Proteins

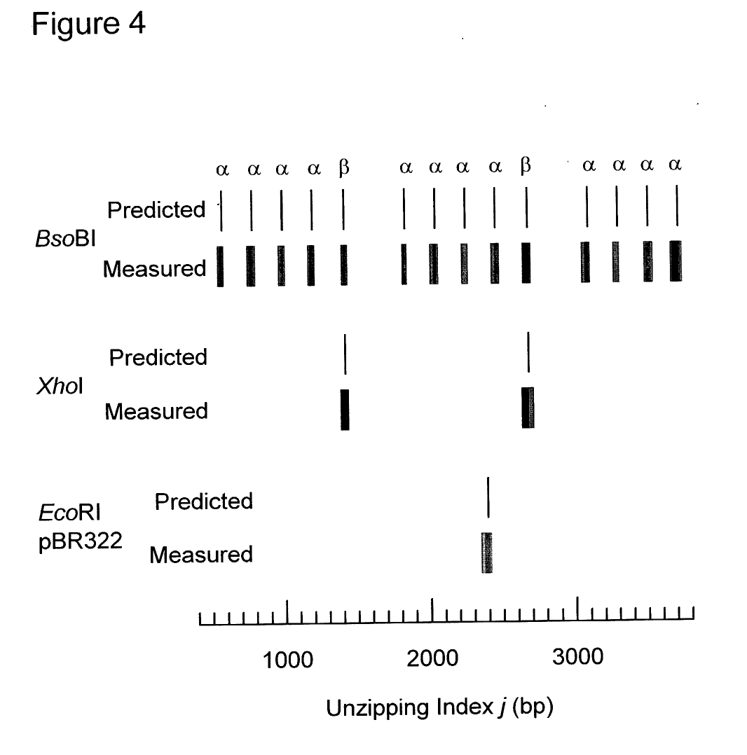

Restriction mapping was used to illustrate one of the important applications of this technique—accurate and precise mapping of bound proteins. Restriction maps were created for three restriction enzymes complexed with the unzipping DNA molecule (FIG. 4, either repetitive or pBR322-derived DNA molecules). EcoRI, BsoBI, and XhoI were disrupted using a proportional velocity clamp at 0.24-0.59 nm nt-1 s-1. BsoBI is known to recognize the sequence CYCGRG (SEQ ID NO:3), where Y is any pyrimidine and R is any purine (Ruan et al., “Cloning and Sequence Comparison of AvaI and BsoBI Restriction-Modification Systems,” Mol. Gen. Genet. 252:695-699 (1996); van der Woerd et al., “Restriction Enzyme Bsobi-DNA Complex. A Tunnel for Recognition of Degenerate DNA Sequences and Potential Histidine Catalysis,” Structure (Camb). 9:133-144 (2001), the entire disclosures of which are hereby incorporated by reference in their entirety). The DNA used to produce FIG. 4 had two different canonical recognition sequences for BsoBI, referred to here as α (ttcCTCGGGaat) (SEQ ID NO:1) and β (aaaCTCGAGaga) (SEQ ID NO:2). The unzipping index axis has been subdivided into bins of 12 bp width, comparable to the footprint of EcoRI and BsoBI as estimated from their crystal structures. Data were combined from stretching several different DNA molecules, so that the grayscale intensity represents the binding fraction of a given bin, i.e., the fraction of the DNA molecules that had unzipping force >25 pN in the bin. These maps show excellent agreement with the expected restriction maps: over a 4 kb DNA molecule, this technique can locate restriction binding sites with an accuracy of ˜25 bp and a precision of 30 bp. This resolution is not expected to decrease appreciably for much longer DNA molecules (for example, DNA molecules of a few Mbp).

With suitable progress towards automation and parallelism, UFAPA could have future applications in the field of genome mapping and sequencing. A recent innovation, optical mapping, is a single-molecule restriction mapping technique that preserves site ordering information (Schwartz et al., “Ordered Restriction Maps of Saccharomyces cerevisiae Chromosomes Constructed by Optical Mapping,” Science 262:110-114 (1993); Cai et al., “High-Resolution Restriction Maps of Bacterial Artificial Chromosomes Constructed by Optical Mapping,” Proc. Natl. Acad. Sci. USA 95:3390-3395 (1998), the entire disclosures of which are hereby incorporated by reference in their entirety). While not suitable for small-scale mapping, the technique was successfully automated to complete a whole-genome shotgun map of a 3 megabase organism (Lin et al., “Whole-Genome Shotgun Optical Mapping of Deinococcus radiodurans,” Science 285:1558-1562 (1999), the entire disclosure of which is hereby incorporated by reference in its entirety). UFAPA shares many of the advantages of optical mapping, including site order preservation as well as other advantages inherent to a single-molecule technique. Furthermore, UFAPA has two other potential advantages—it is a non-imaging technique, allowing for better base pair location resolution, and UFAPA is non-catalytic, allowing for reversible rapid screening of multiple enzymes on a single molecule.

### Example 9

### Determination of Equilibrium Association Constants

The affinity of a protein for its DNA binding site is characterized by the equilibrium association constant. Since UFAPA can directly detect protein-DNA binding, it allows for, direct, and site-specific measurements of equilibrium association constants, K_{A, XY}:

$$\begin{matrix}{protein}_{X} + {DNA}_{site Y} ↔ {protein}_{X} \cdot {DNA}_{site Y} , & ( 1 ) \\ and & \\ {K}_{A , XY} = \frac{1}{[ {protein}_{X} ]} \frac{[ {protein}_{X} \cdot {DNA}_{site Y} ]}{[ {DNA}_{site Y} ]} \cdot & ( 2 )\end{matrix}$$

For a given protein X concentration, the ratio of bound to unbound Y sites,

$$r = \frac{[ {protein}_{X} \cdot {DNA}_{Site Y} ]}{[ {DNA}_{site Y} ]} ,$$

gives a measure of the equilibrium association constant K_{A, XY}. As in a gel-mobility shift assay, and unlike filter-binding assays, measurements at a single protein concentration are sufficient to determine K_{A}, although in general, titration of protein could be useful to reveal deviations from the above relation and to determine binding stoichiometry. The free EcoRI concentration was the same as the total protein concentration (50 to 6000 pM) due to the low effective DNA concentration (≦3 pM).

The distribution of the number of bound sites follows a binomial distribution, the relative uncertainty in K_{A} is as follows:

$$\frac{{σ}_{{K}_{A}}}{{K}_{A}} = \frac{1 + r}{\sqrt{r ( N - 1 )}} , \qquad ( 3 )$$

where σ_{K}_{_{A}} is the standard deviation and N is the number of measurements. Eq. 3 shows that for a given number of measurements, the best precision is obtained when r=1. For the present study, r was kept near 1, in order to minimize the error; other than increased uncertainty, no differences in the mean values of K_{A} were observed when r≅0.1 or r≅10.

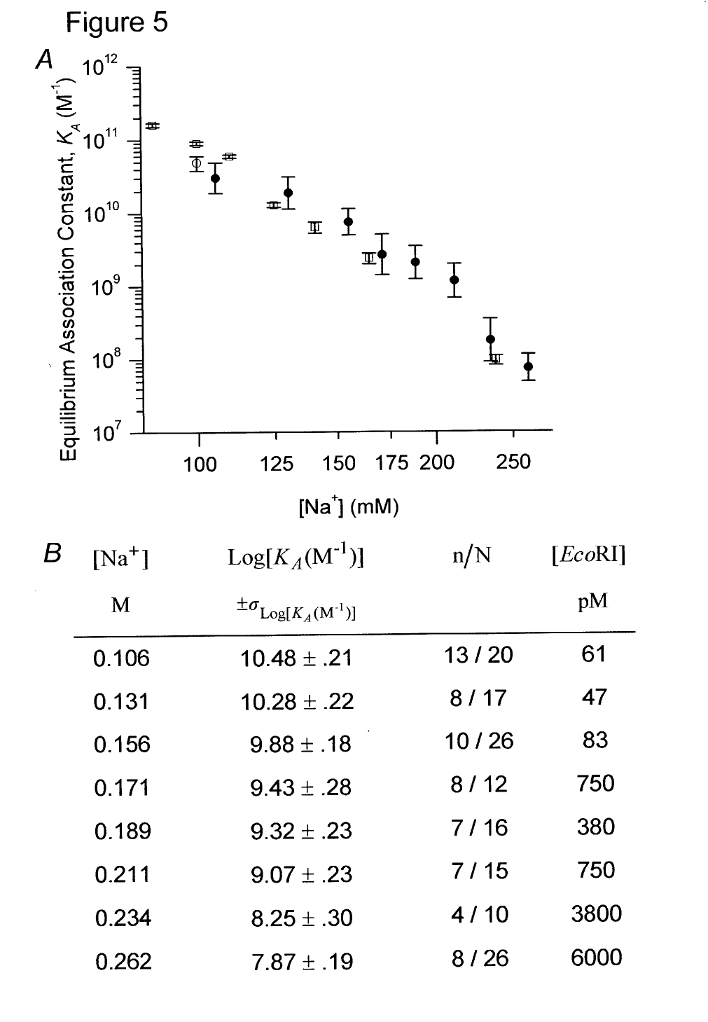

FIG. 5A shows K_{A} values that were determined for EcoRI binding to its canonical site on pBR322 at various Na+ concentrations (solid dots). Data in FIG. 5A are also tabulated in FIG. 5B. For a given DNA molecule, a site was considered bound if the unzipping force exceeded 20 pN within 100 bp of the expected site. By making measurements at a given site on multiple DNA molecules, the ratio of bound to unbound sites, r, was obtained for that site. In FIG. 5, both the K_{A} values and their error bars are shown on a logarithmic scale. For N DNA molecules probed and n bound enzymes detected, the standard deviation in log(K_{A}) is given by:

$${τ}_{log ( {K}_{A} )} = {log ( e ) [ ( N - 1 ) ( 1 - \frac{n}{N} ) \frac{n}{N} ]}^{- 1 / 2}$$

based on a binomial distribution.

For comparison, FIG. 5A also shows K_{A} values for EcoRI binding to its canonical site on pBR322 at various Na+ concentrations from Ha et al., “Role of the Hydrophobic Effect in Stability of Site-Specific Protein-DNA Complexes,” J. Mol. Biol. 209:801-816 (1989), the entire disclosure of which is hereby incorporated by reference in its entirety (open circle) and Terry et al., “Thermodynamic Parameters Governing Interaction of EcoRI Endonuclease with Specific and Nonspecific DNA Sequences,” J. Biol. Chem. 258:9820-9825 (1983), the entire disclosure of which is hereby incorporated by reference in its entirety (open squares). The exact conditions for these measurements are given in the figure caption. The buffers are essentially the same as those used by Ha et al., “Role of the Hydrophobic Effect in Stability of Site-Specific Protein-DNA Complexes,” J. Mol. Biol. 209:801-816 (1989), the entire disclosure of which is hereby incorporated by reference in its entirety, and have the same pH as those used by Terry et al., “Thermodynamic Parameters Governing Interaction of EcoRI Endonuclease with Specific and Nonspecific DNA Sequences,” J. Biol. Chem. 258:9820-9825 (1983), the entire disclosure of which is hereby incorporated by reference in its entirety. The measurement temperature (23° C.) was similar to that of Ha et al., “Role of the Hydrophobic Effect in Stability of Site-Specific Protein-DNA Complexes,” J. Mol. Biol. 209:801-816 (1989), the entire disclosure of which is hereby incorporated by reference in its entirety (21.1° C.), but somewhat different from that of Terry et al., “Thermodynamic Parameters Governing Interaction of EcoRI Endonuclease with Specific and Nonspecific DNA Sequences,” J. Biol. Chem. 258:9820-9825 (1983), the entire disclosure of which is hereby incorporated by reference in its entirety (37° C.).

The UFAPA measurements in FIG. 5 overlap the values of Terry et al., “Thermodynamic Parameters Governing Interaction of EcoRI Endonuclease with Specific and Nonspecific DNA Sequences,” J. Biol. Chem. 258:9820-9825 (1983), the entire disclosure of which is hereby incorporated by reference in its entirety, over most of the [Na+] ranging from 131 to 262 mM. Deviation from the data of Terry et al., “Thermodynamic Parameters Governing Interaction of EcoRI Endonuclease with Specific and Nonspecific DNA Sequences,” J. Biol. Chem. 258:9820-9825 (1983), the entire disclosure of which is hereby incorporated by reference in its entirety, at the lower salt condition, [Na+]=106 mM, is most likely due to protein aggregation, which is known to occur for EcoRI at low ionic strength, and in the absence of saturating DNA binding sites (Jen-Jacobson et al., “Coordinate Ion Pair Formation Between EcoRI Endonuclease and DNA,” J. Biol. Chem. 258:14638-14646 (1983), the entire disclosure of which is hereby incorporated by reference in its entirety). Terry et al. (Terry et al., “Thermodynamic Parameters Governing Interaction of EcoRI Endonuclease with Specific and Nonspecific DNA Sequences,” J. Biol. Chem. 258:9820-9825 (1983), the entire disclosure of which is hereby incorporated by reference in its entirety) found the slope of their data to be commensurate with 8 ion pairs involved in the binding of EcoRI to the pBR322 site—a result consistent with that of Jen-Jacobson et al., “Coordinate Ion Pair Formation Between EcoRI Endonuclease and DNA,” J. Biol. Chem. 258:14638-14646 (1983), the entire disclosure of which is hereby incorporated by reference in its entirety. Over the [Na+] range of 131 to 234 mM, UFAPA data follow a similar slope to Terry et al., “Thermodynamic Parameters Governing Interaction of EcoRI Endonuclease with Specific and Nonspecific DNA Sequences,” J. Biol. Chem. 258:9820-9825 (1983), the entire disclosure of which is hereby incorporated by reference in its entirety. It is currently unknown whether the apparent increase in slope magnitude around 262 mM Na+ is due to technical difficulties of the UFAPA method, although the filter binding assay from Terry et al., “Thermodynamic Parameters Governing Interaction of EcoRI Endonuclease with Specific and Nonspecific DNA Sequences,” J. Biol. Chem. 258:9820-9825 (1983), the entire disclosure of which is hereby incorporated by reference in its entirety, shows a similar effect.

Determination of K_{A} using UFAPA offers a number of new features compared with traditional bulk equilibrium methods. UFAPA is direct and site specific, reducing possible complications from non-specific DNA binding sometimes encountered in bulk studies. A single UFAPA measurement is fast, avoiding possible dissociation of the protein-DNA complex before a measurement is obtained as may occur in bulk studies (for example, while the sample is entering a gel). Future studies may elucidate whether UFAPA is useful to probe K_{A} values with particularly fast dissociation rates. Values of K_{A} can be determined simultaneously for multiple protein-DNA interactions at different binding sites on the DNA.

Determination of K_{A} using UFAPA also has some limitations. The principle limitation of the UFAPA approach is the lack of commercially available low-cost instrumentation with suitable automation for precise counting statistics. As shown in FIG. 5, counting binding from a few molecules results in a large uncertainty: 10 counts produces at best 67% precision, and 400 counts are required to achieve at best 10% precision for a level consistent with the results of Terry et al., “Thermodynamic Parameters Governing Interaction of EcoRI Endonuclease with Specific and Nonspecific DNA Sequences,” J. Biol. Chem. 258:9820-9825 (1983), the entire disclosure of which is hereby incorporated by reference in its entirety. Established biochemical assays also have the advantage of running many samples in parallel: up to as many lanes or filter ports are available on the apparatus. A further point to consider is the current inability to easily titrate the concentration of DNA binding sites. In the current implementation, the DNA is surface tethered and there is no ability to perform assays under saturating DNA conditions. Future enhancements may allow the immobilization of the DNA-binding protein, and subsequent titration of DNA against various protein surface densities. These enhancements would allow determination of binding activity, oligomeric state of binding protein, and other information.

As with traditional biochemical studies of K_{A}, UFAPA also has a range of accessible K_{A} values. The lower limit will depend on the solubility of the particular protein, and the ability to have enough protein in solution to have appreciable ratio of bound sites. The upper limit for UFAPA can in principle be raised higher than the ˜1011 M-1 measured in this report. The current implementation requires that there be significantly fewer DNA binding sites than protein molecules so that the DNA's alteration of the free protein concentration is negligible. As K_{A} increases, the amount of surface-tethered DNA should also be decreased. At some point, the reduction in surface tether density would make the current practice unmanageable but values of at least 1012 M-1 are expected to be measurable by lowering the effective DNA concentration (<0.1 pM). These potential limits are not strictly defined and future enhancements of UFAPA could expand the accessible K_{A} range, although it is not clear whether this range would exceed that spanned by established bulk assays.

### Example 10

### Dynamic Force Spectroscopy of Protein-DNA Interactions by Unzipping DNA.

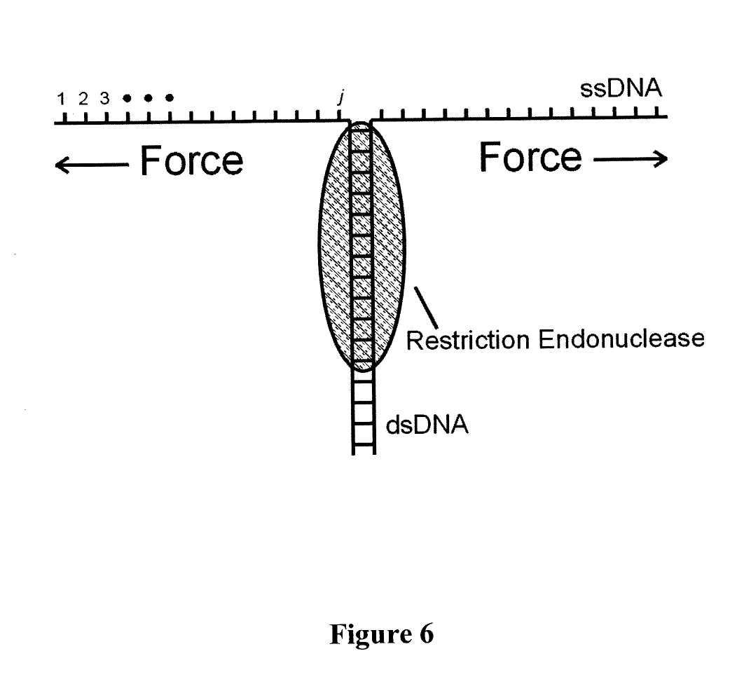

FIG. 6 is a schematic showing the general principle of UFAPA. A bound restriction enzyme further reduces the free energy of the stable complex (hydrogen bonds, vanderwaals and hydrophobic interactions) beyond the normal base pairing and stacking energy. The feedback for data acquisition was tailored for the purposes of dynamic force spectroscopy. A feedback system with real-time calculation of unzipping index, j, was implemented so as to disrupt the protein-DNA complexes with a force increasing linearly with time (loading rate clamp).

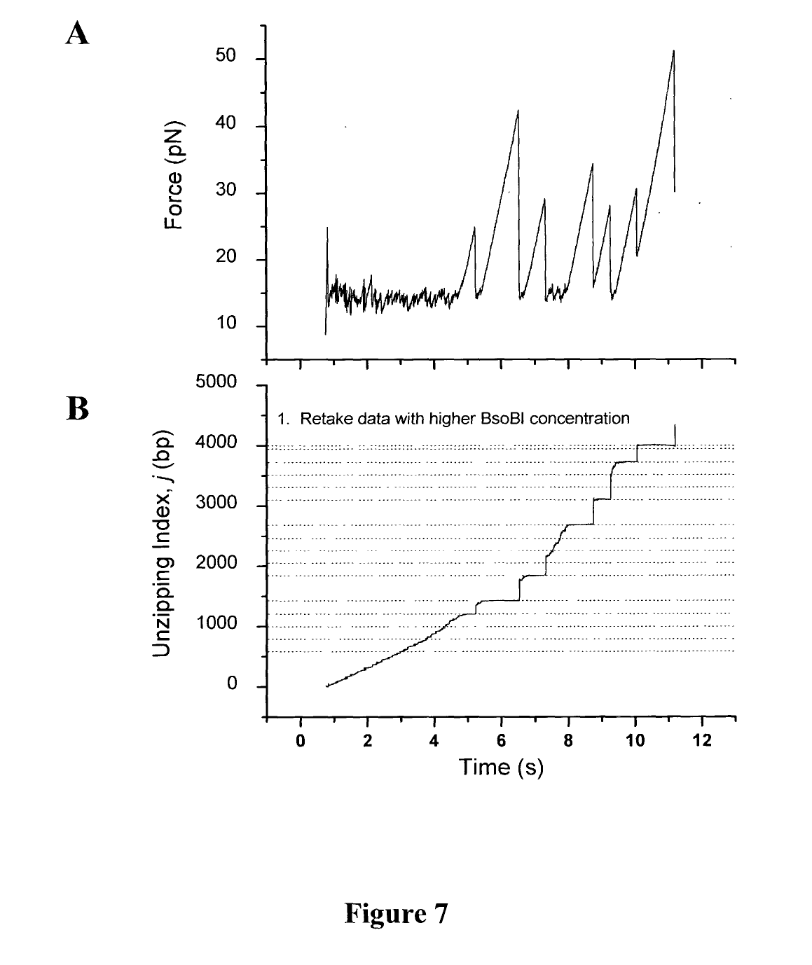

FIG. 7A shows data taken with the loading rate clamp, and the resulting linear force versus time curves for BsoBI unbinding from the pCP681 derived DNA construct. The time periods when the restriction enzyme was pinning the double helix are seen in FIG. 7A as the linear slopes when force is between ˜15 pN and the ultimate disruption force (25-50 pN in the figure). The event time periods are also revealed in FIG. 7B as horizontal plateaus for the unzipping index, j, which was calculated as described previously (Koch et al., “Probing Protein-DNA Interactions by Unzipping a Single DNA Double Helix,” Biophys. J. 83:1098-1105 (2002), the entire disclosure of which is hereby incorporated by reference in its entirety).

The benefits of the loading rate clamp are two fold, when compared with a velocity clamp or proportional velocity clamp. First, a linear force loading rate greatly simplifies the theoretical treatment of the data (Evans et al., “Probing the Relation Between Force—Lifetime—and Chemistry in Single Molecular Bonds,” Annu. Rev. Biophys. Biomol. Struct. 30:105-128 (2001), the entire disclosure of which is hereby incorporated by reference in its entirety). Second, the linear force loading rate produces unbinding force distributions with a smaller thermodynamic spread.

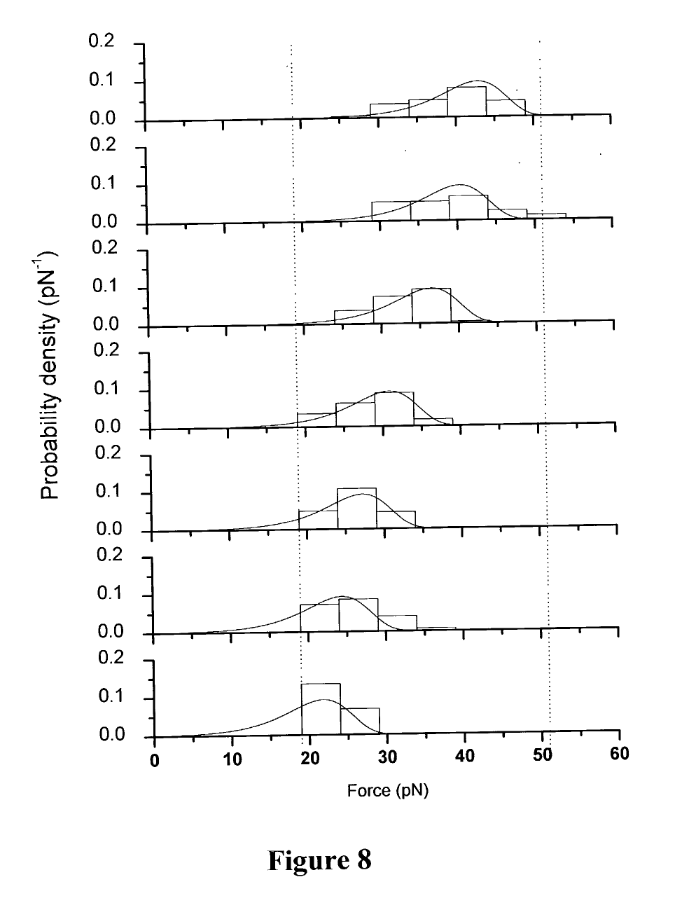

As shown in FIG. 7, unbinding events are easily distinguished from the baseline unzipping forces. An automated event detection scheme locates each event, and determines an event starting force, the disruption force, and the average loading rate during the event. Under the action of the loading rate clamp, the observed loading rate varies by less than about 25% (standard deviation of 20 ms time periods). Within the context of dynamic force spectroscopy (“DFS”), this spread is insignificant, due to the logarithmic relationship between the expected force distribution and the force loading rate (Evans et al., “Probing the Relation Between Force—Lifetime—and Chemistry in Single Molecular Bonds,” Annu. Rev. Biophys. Biomol. Struct. 30:105-128 (2001), the entire disclosure of which is hereby incorporated by reference in its entirety). In FIG. 8, a summary of BsoBI unbinding from a sites is shown. Data from unzipping many individual DNA constructs are grouped by loading rate, and binned into histograms. The loading rate for a given group is the simple mean of the loading rates.

A maximum likelihood method (Bevington et al., “Direct Application of the Maximum Likelihood Method,” Data Reduction and Error Analysis for the Physical Sciences 0:1-2 (1992), Chapter 10, the entire disclosure of which is hereby incorporated by reference in its entirety) is employed to directly fit the data in FIG. 8 to an overall probability distribution

$$p ∝ \frac{{f}_{β}}{{t}_{off} \cdot r} \cdot exp ( f \text{/} {f}_{β} ) \cdot exp ( \frac{{f}_{β}}{{t}_{off} \cdot r} ) \cdot exp ( - exp ( f \text{/} {f}_{β} ) \cdot \frac{{f}_{β}}{{t}_{off} \cdot r} ) ,$$

from Evans et al., “Probing the Relation Between Force—Lifetime—and Chemistry in Single Molecular Bonds,” Annu. Rev. Biophys. Biomol. Struct. 30:105-128 (2001), the entire disclosure of which is hereby incorporated by reference in its entirety. In this equation, f is the event unbinding force, r is the event loading rate (pN/s), and f_{β} and t_{off} are parameters describing the simple energy well. This distribution assumes that all forces from 0 to ∞ are experimentally accessible, whereas the present invention uses both a lower and an upper force cutoff. The lower force threshold depends on the starting force for the particular event, while the upper cutoff force is set to 51 pN, to prevent overstretching of the DNA handle (Koch et al., “Probing Protein-DNA Interactions by Unzipping a Single DNA Double Helix,” Biophys. J. 83:1098-1105 (2002), the entire disclosure of which is hereby incorporated by reference in its entirety). To account for this, the above expression is modified to set p(f) to zero outside of the experimental range, and normalize the remaining probability so the integral remains unity.

To find global fit parameters, a Nelder-Mead simplex method is utilized to search for the maximum of the natural logarithm of the likelihood estimator (Bevington et al., “Direct Application of the Maximum Likelihood Method,” Data Reduction and Error Analysis for the Physical Sciences 0:1-2 (1992), Chapter 10, the entire disclosure of which is hereby incorporated by reference in its entirety). The resulting global fit is shown as solid lines over the data in FIG. 8. It can be seen that for BsoBI unbinding from α sites, the actual measured unbinding distributions obey the simple model proposed by DFS across the loading rates investigated. The same fitting method applied to BsoBI unbinding from β sites and XhoI unbinding from β sites combined with the BsoBI α data are summarized in Table 1.

TABLE 1

| Enzyme | Site | F_{β} | ln(t_{off}(s)) | N |
|---|---|---|---|---|
| BsoBI | ttcCTCGGGaat | 4.04 ± 0.14 pN | 4.98 ± 0.27 | 243 |
| | (SEQ ID NO:1) | | | |
| BsoBI | aaaCTCGAGaga | 4.36 ± 0.53 pN | 7.6 ± 1.1 | 51 |
| | (SEQ ID NO:2) | | | |
| XhoI | aaaCTCGAGaga | 3.20 ± 0.30 pN | 9.9 ± 1.1 | 41 |
| | (SEQ ID NO:2) | | | |

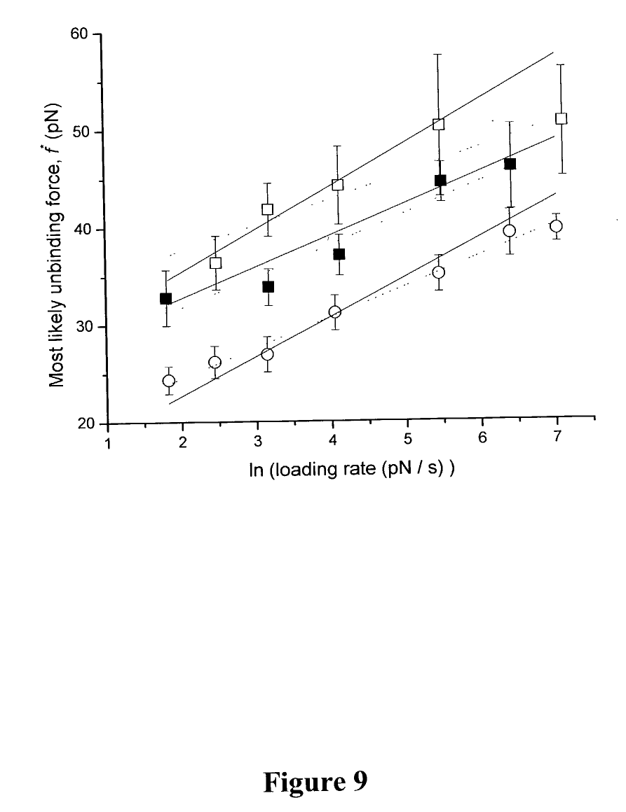

It is notable that in Table 1 all the three different binding species show a different DFS signature. This is more clearly represented in FIG. 9, where the data are presented in the traditional f* versus ln(r) form. The data in FIG. 9 are obtained from sliding Gaussian window histograms, and the error bars represent the standard deviation obtained from 1,000 Monte Carlo simulations at the same stretch rate and the same parameters from Table 1. The dotted lines in FIG. 9 represent least-squares fits to the data, as would traditionally be performed in a DFS study (Evans et al., “Probing the Relation Between Force—Lifetime—and Chemistry in Single Molecular Bonds,” Annu. Rev. Biophys. Biomol. Struct. 30:105-128 (2001), the entire disclosure of which is hereby incorporated by reference in its entirety). The solid lines shown in FIG. 9 are not linear fits to the data, but instead the predicted f* behavior, f^{*}=f_{β}·ln(r·t_{off}/f_{β}), using the maximum likelihood fit parameters (Table 1). It can be seen that the dotted lines and the solid lines are different, which may be due to the difficulty and bias encountered with the traditional DFS method.

While the direct maximum likelihood fitting can extract the DFS fit parameters, it is still useful to use FIG. 9 to illustrate the distinct binding signatures of the three different complexes and to determine the loading rates at which to examine differences between binding species, as shown below.

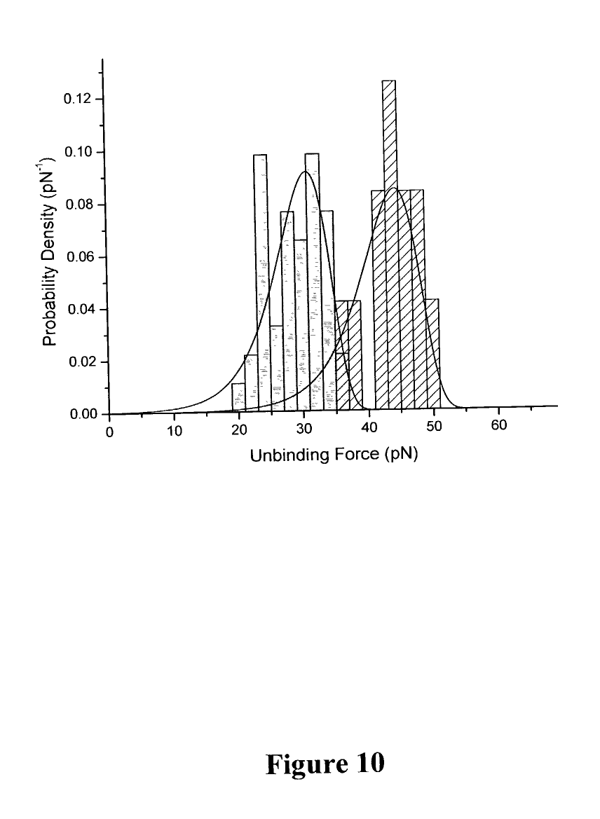

An important result shown in Table 1 and FIG. 9 is that the 3 classes of binding species are distinguishable from each other: The same protein (BsoBI) binding to two different sites (α versus β) produces dramatically different unbinding force distributions. Similarly, two different proteins (BsoBI and XhoI) binding to the same DNA site (β) produce distinguishable binding signatures. This is illustrated more clearly in FIG. 10, where unbinding force distributions are shown for BsoBI unbinding from α (filled bars) and β (lined bars) sites at a rate of ˜60 pN/s. FIG. 10 shows clearly distinguishable distributions.

The UFAPA technique presented here is a general tool for detection of protein-DNA interactions. It is a single molecule technique that yields the locations of bound proteins and the equilibrium association constants for the protein-DNA interactions. As further enhancements are made, there should be broad applications of UFAPA in the study of protein-DNA interactions from simple binding site detection and DNA sequence analysis, to the determination of previously unknown protein binding sites on DNA and the detection of previously unknown DNA-binding proteins.

The DFS part of UFAPA presents a powerful tool for studying protein-DNA interactions. There are two clear implications. Further quantitative studies are possible if it can be shown that the DFS fit parameters, particularly t_{off}, can be tied to relevant parameters of the interaction. For example, if t_{off} can be shown in some cases to be equivalent or proportional to the actual off rate for site-specific binding (as compared with the much more easily measurable overall off rate), then UFAPA would present the only currently available method for directly determining this rate constant. Further, with careful analysis of the unbinding forces, it may be possible to distinguish between binding species on a molecule-by-molecule basis. This ability should prove critical for future single-molecule UFAPA applications, for example, competition binding assays, or simultaneous multiple restriction enzyme.

Although preferred embodiments have been depicted and described in detail herein, it will be apparent to those skilled in the relevant art that various modifications, additions, substitutions and the like can be made without departing from the spirit of the invention and these are therefore considered to be within the scope of the invention as defined in the claims which follow.

What is claimed:

1. A method of identifying the location of a binding site for a binding molecule on a double-stranded nucleic acid molecule, said method comprising:
providing a double-stranded nucleic acid molecule comprising first and second nucleic acid strands;
providing a binding molecule;
contacting the double-stranded nucleic acid molecule and the binding molecule with one another under conditions effective to yield a modified nucleic acid molecule comprising the binding molecule bound to the double-stranded nucleic acid molecule;
unzipping the first and second nucleic acid strands from one another under conditions effective to disrupt the binding molecule, if any, bound to the double-stranded nucleic acid molecule; and
identifying binding site locations, if any, for the binding molecule on the nucleic acid molecule by comparing force required to unzip the first and second nucleic acid strands from one another with the force required to unzip the nucleic acid molecule with no binding molecule bound thereto, wherein locations with a change in the force required to unzip the modified nucleic acid molecule compared to the force required to unzip the nucleic acid molecule with no binding molecule bound thereto are binding sites on the modified nucleic acid molecule.

2. The method according to claim 1 further comprising:
securing a 5′-portion of the first nucleic acid strand to a first securing component prior to said unzipping and
securing a 3′-portion of the second nucleic acid strand to a second securing component prior to said unzipping.

3. The method according to claim 2, wherein said securing is achieved using a nucleic acid bonding technique.

4. The method according to claim 3, wherein said nucleic acid bonding technique is selected from the group consisting of streptavidin/biotin binding and antibody/antigen binding.

5. The method according to claim 2, wherein said first and second securing components are selected from the group consisting of a microwell, a microtiter plate, a microscope slide, a miscroscope coverslip, a microsphere, a column, a disc, a membrane, a film, a micropipette, a nanotube, a tip of an optical fiber, and a tip of a scanning probe.

6. The method according to claim 2, wherein said unzipping comprises either of the following:
moving the first securing component away from the second securing component, wherein said second securing component is kept relatively stationary;
moving the second securing component away from the first securing component, wherein said second securing component is kept relatively stationary; or
simultaneously moving the first and second securing component away from one another.

7. The method according to claim 6, wherein motion or lack of motion of said first or second securing component is achieved by using a technique selected from the group consisting of optical trapping technology, micropipette technology, viscous drag force, atomic force microscopy, magnetic force microscopy, optical fiber force transducer technology, nano-fabricated cantilever or tip technology, micro-fabricated cantilever or tip technology, nanotube technology, and microelectromechanical technology.

8. The method according to claim 1, wherein the change in force is either an increase or decrease in the force required to unzip the first and second nucleic acid strands of the modified nucleic acid molecule compared to the force required to unzip the nucleic acid molecule with no binding molecule bound thereto.

9. The method according to claim 1, wherein said identifying comprises:
measuring force using force sensor technology.

10. The method according to claim 9, wherein the force sensor technology is selected from the group consisting of optical trapping technology, micropipette technology, viscous drag force, atomic force microscopy, magnetic force microscopy, nano-fabricated cantilever or tip technology, micro-fabricated cantilever or tip technology, nanotube technology, optical fiber force transducer technology, and microelectromechanical technology.

11. The method according to claim 10, wherein the force sensor technology is optical trapping technology comprising a feedback-enhanced optical trap.

12. The method according to claim 1, wherein the binding molecule is selected from the group consisting of a polypeptide, an oligonucleotide, an inorganic chemical compound, an organic chemical compound, and a PNA.

13. The method according to claim 12, wherein the binding molecule is a polypeptide selected from the group consisting of a restriction endonuclease, a polymerase, a helicase, a nuclease, an isomerase, a ligase, activators, repressors, and a histone.

14. The method according to claim 1, wherein said double-stranded nucleic acid molecule is selected from the group consisting of a double-stranded DNA molecule, a double-stranded RNA molecule, and a DNA/RNA duplex molecule.

15. A method of directly determining an equilibrium association constant of a target binding molecule specific to a double-stranded nucleic acid molecule, said method comprising:
(a) providing a double-stranded nucleic acid molecule suspected of having a binding site for the target binding molecule, said double-stranded nucleic acid molecule comprising first and second nucleic acid strands;
(b) providing the target binding molecule;
(c) contacting the double-stranded nucleic acid molecule and the target binding molecule with one another under conditions effective to yield a greater than 0% and less than 100% chance that a modified nucleic acid molecule comprising the target binding molecule bound to the double-stranded nucleic acid molecule will be produced;
(d) unzipping the first and second nucleic acid strands from one another under conditions effective to disrupt the target binding molecule, if any, bound to the double-stranded nucleic acid molecule;
(e) determining whether a binding site on the double stranded nucleic acid molecule is occupied by the target binding molecule by comparing force required to unzip the first and second nucleic acid strands from one another with force required to unzip the double-stranded nucleic acid molecule having no target binding molecule bound thereto, wherein a change in force required to unzip the double-stranded nucleic acid molecule having no target binding molecule indicates the presence of a target binding molecule;
(f) repeating steps (a) through (e) at least one time; and
(g) calculating a ratio of occupied to unoccupied binding sites as a result of carrying out steps (a) through (f) and dividing the ratio by bulk protein concentration bound to unbound target binding molecules to the suspected binding site based on the determination made in step (e), said ratio being an equilibrium constant of the target binding molecule to the suspected binding site.

16. The method according to claim 15 further comprising:
securing a 5′-portion of the first nucleic acid strand to a first securing component prior to said unzipping and
securing a 3′-portion of the second nucleic acid strand to a second securing component prior to said unzipping.

17. The method according to claim 16, wherein said securing is achieved using a nucleic acid bonding technique.

18. The method according to claim 17, wherein said nucleic acid bonding technique is selected from the group consisting of streptavidin/biotin binding and antibody/antigen binding.

19. The method according to claim 16, wherein said first and second securing components are selected from the group consisting of a microwell, a microtiter plate, a microscope slide, a miscroscope coverslip, a microsphere, a column, a disc, a membrane, a film, a micropipette, a nanotube, a tip of an optical fiber, and a tip of a scanning probe.

20. The method according to claim 16, wherein said unzipping comprises either of the following:
moving the first securing component away from the second securing component, wherein said second securing component is kept relatively stationary;
moving the second securing component away from the first securing component, wherein said second securing component is kept relatively stationary; or
simultaneously moving the first and second securing component away from one another.

21. The method according to claim 20, wherein motion or lack of motion of said first or second securing component is achieved by using a technique selected from the group consisting of optical trapping technology, micropipette technology, viscous drag force, atomic force microscopy, magnetic force microscopy, optical fiber force transducer technology, nano-fabricated cantilever or tip technology, micro-fabricated cantilever or tip technology, nanotube technology, and microelectromechanical technology.

22. The method according to claim 15, wherein said determining comprises:
measuring force using force sensor technology.

23. The method according to claim 22, wherein the force sensor technology is selected from the group consisting of optical trapping technology, micropipette technology, viscous drag force, atomic force microscopy, magnetic force microscopy, nano-fabricated cantilever or tip technology, micro-fabricated cantilever or tip technology, nanotube technology, optical fiber force transducer technology, and microelectromechanical technology.

24. The method according to claim 27, wherein the force sensor technology is optical trapping technology comprising a feedback-enhanced optical trap.

25. The method according to claim 19, wherein the binding molecule is selected from the group consisting of a polypeptide, an oligonucleotide, an inorganic chemical compound, an organic chemical compound, and a PNA.

26. The method according to claim 29, wherein the binding molecule is a polypeptide selected from the group consisting of a restriction endonuclease, a polymerase, a helicase, a nuclease, an isomerase, a ligase, activators, repressors, and a histone.

27. The method according to claim 19, wherein said double-stranded nucleic acid molecule is selected from the group consisting of a double-stranded DNA molecule, a double-stranded RNA molecule, and a DNA/RNA duplex molecule.

28. A method of determining the dynamic force signature of a target binding molecule in relation to a binding site on a double-stranded nucleic acid molecule, said method comprising:
(a) providing a double-stranded nucleic acid molecule suspected of having a binding site for the target binding molecule, said double-stranded nucleic acid molecule comprising first and second nucleic acid strands and;
(b) providing the target binding molecule;
(c) contacting the double-stranded nucleic acid molecule and the target binding molecule with one another under conditions effective to yield a modified nucleic acid molecule having at least one binding complex comprising the target binding molecule bound to the double-stranded nucleic acid molecule;
(d) unzipping the first and second nucleic acid strands from one another under conditions effective to disrupt the target binding molecule, if any, bound to the double-stranded nucleic acid molecule;
(e) determining unzipping location, starting force, peak force for disruption, and force loading pattern for the target binding molecule;
(f) repeating steps (a) through (e); and
(g) calculating the dynamic force signature for the binding molecule from the unzipping location, the starting force, the peak force, and the force loading pattern determinations.

29. The method according to claim 28 further comprising:
securing a 5′-portion of the first nucleic acid strand to a first securing component prior to said unzipping and
securing a 3′-portion of the second nucleic acid strand to a second securing component prior to said unzipping.

30. The method according to claim 29, wherein said securing is achieved using a nucleic acid bonding technique.

31. The method according to claim 30, wherein said nucleic acid bonding technique is selected from the group consisting of streptavidin/biotin binding and antibody/antigen binding.

32. The method according to claim 29, wherein said first and second securing components are selected from the group consisting of a microwell, a microtiter plate, a microscope slide, a miscroscope coverslip, a microsphere, a column, a disc, a membrane, a film, a micropipette, a nanotube, a tip of an optical fiber, and a tip of a scanning probe.

33. The method according to claim 29, wherein said unzipping comprises either of the following:
moving the first securing component away from the second securing component, wherein said second securing component is kept relatively stationary;
moving the second securing component away from the first securing component, wherein said second securing component is kept relatively stationary; or
simultaneously moving the first and second securing component away from one another.

34. The method according to claim 33, wherein motion or lack of motion of said first or second securing component is achieved using a technique selected from the group consisting of optical trapping technology, micropipette technology, viscous drag force, atomic force microscopy, magnetic force microscopy, optical fiber force transducer technology, nano-fabricated cantilever or tip technology, micro-fabricated cantilever or tip technology, nanotube technology, and microelectromechanical technology.

35. The method according to claim 28, wherein said determining comprises:
measuring force using force sensor technology.

36. The method according to claim 35, wherein the force sensor technology is selected from the group consisting of optical trapping technology, micropipette technology, viscous drag force, atomic force microscopy, magnetic force microscopy, nano-fabricated cantilever or tip technology, micro-fabricated cantilever or tip technology, nanotube technology, optical fiber force transducer technology, and microelectromechanical technology.

37. The method according to claim 36, wherein the force sensor technology is optical trapping technology comprising a feedback-enhanced optical trap.

38. The method according to claim 28, wherein the binding molecule is selected from the group consisting of a polypeptide, an oligonucleotide, an inorganic chemical compound, an organic chemical compound, and a PNA.

39. The method according to claim 38, wherein the binding molecule is a polypeptide selected from the group consisting of a restriction endonuclease, a polymerase, a helicase, a nuclease, an isomerase, a ligase, activators, repressors, and a histone.

40. The method according to claim 28, wherein said double-stranded nucleic acid molecule is selected from the group consisting of a double-stranded DNA molecule, a double-stranded RNA molecule, and a DNA/RNA duplex molecule.

41. The method according to claim 28, wherein the target binding molecule is a drug candidate which either binds to the double-stranded nucleic acid molecule or inhibits binding of another material to the double-stranded nucleic acid molecule.

42. A method of identifying whether a target nucleic acid molecule is present in a sample, said method comprising:
providing a sample potentially containing a double-stranded target nucleic acid molecule comprising first and second nucleic acid strands;
providing a protein which binds to a binding site on the target nucleic acid molecule;
contacting the sample and the protein with one another under conditions effective to permit the protein to bind to the target nucleic acid molecule, if present in the sample;
unzipping the first and second nucleic acid strands, if present, from one another under conditions effective to separate the first and second nucleic acid strands; and
identifying a presence of any target nucleic acid molecules by measuring force required to unzip the first and second nucleic acid strands from one another, wherein a change in force indicates the presence of the target nucleic acid molecule in the sample.

43. The method according to claim 42, further comprising:
securing a 5′-portion of the first nucleic acid strand to a first securing component prior to said unzipping and
securing a 3′-portion of the second nucleic acid strand to a second securing component prior to said unzipping.

44. The method according to claim 43, wherein said securing is achieved using a nucleic acid bonding technique.

45. The method according to claim 44, wherein said nucleic acid bonding technique is selected from the group consisting of streptavidin/biotin binding and antibody/antigen binding.

46. The method according to claim 43, wherein said first and second securing components are selected from the group consisting of a microwell, a microtiter plate, a microscope slide, a miscroscope coverslip, a microsphere, a column, a disc, a membrane, a film, a micropipette, a nanotube, a tip of an optical fiber, and a tip of a scanning probe.

47. The method according to claim 43, wherein said unzipping comprises either of the following:
moving the first securing component away from the second securing component, wherein said second securing component is kept relatively stationary;
moving the second securing component away from the first securing component, wherein said second securing component is kept relatively stationary; or
simultaneously moving the first and second securing component away from one another.

48. The method according to claim 47, wherein motion or lack of motion of said first or second securing component is achieved by using a technique selected from the group consisting of optical trapping technology, micropipette technology, electromagnetic force, atomic force microscopy, magnetic force microscopy, optical fiber force transducer technology, mechanical technology, nano-fabricated cantilever or tip technology, micro-fabricated cantilever or tip technology, nanotube technology, and microelectromechanical technology.

49. The method according to claim 42, wherein said identifying comprises:
measuring force using force sensor technology.

50. The method according to claim 49, wherein the force sensor technology is selected from the group consisting of optical trapping technology, micropipette technology, electromagnetic force, atomic force microscopy, magnetic force microscopy, nano-fabricated cantilever or tip technology, micro-fabricated cantilever or tip technology, nanotube technology, optical fiber force transducer technology, and microelectromechanical technology.

51. The method according to claim 50, wherein the force sensor technology is optical trapping technology comprising a feedback-enhanced optical trap.

52. The method according to claim 42, wherein the binding molecule is selected from the group consisting of a polypeptide, an oligonucleotide, an inorganic chemical compound, an organic chemical compound, and a PNA.

53. The method according to claim 52, wherein the binding molecule is a polypeptide selected from the group consisting of a restriction endonuclease, a polymerase, a helicase, a nuclease, an isomerase, a ligase, activators, repressors, and a histone.

54. The method according to claim 42, wherein said double-stranded nucleic acid molecule is selected from the group consisting of a double-stranded DNA molecule, a double-stranded RNA molecule, and a DNA/RNA duplex molecule.

55. A method of producing a restriction map for a nucleic acid molecule, said method comprising:
(a) providing a double-stranded nucleic acid molecule comprising first and second nucleic acid strands;
(b) providing a restriction endonuclease;
(c) contacting the double-stranded nucleic acid molecule and the restriction endonuclease with one another under conditions effective to yield a binding complex comprising the restriction endonuclease bound to the double-stranded nucleic acid molecule, while prohibiting said restriction endonuclease from cutting the nucleic acid molecule;
(d) unzipping the first and second nucleic acid strands from one another under conditions effective to separate the first and second nucleic acid strands from one another; and
(e) identifying binding site locations, if any, for the restriction endonuclease on the nucleic acid molecule by comparing force required to unzip the first and second nucleic acid strands from one another with force required to unzip the double-stranded nucleic acid molecule having no restriction endonuclease bound thereto, wherein a change in force required to unzip the double-stranded nucleic acid molecule having no restriction endonuclease indicates the location of a binding site for the restriction endonuclease on the double-stranded nucleic acid molecule.

56. The method according to claim 55 further comprising:
repeating steps (a) through (e) for each additional restriction endonuclease to be mapped.

57. The method according to claim 56, wherein the restriction endonuclease is selected from the group consisting of EcoRI, BsoBI, XhoI, BamHI, HaeIII, HpaI, PstI, Sau3A, SamI, SstI, XmaI, and combinations thereof

58. The method according to claim 57 further comprising:
securing a 5′-portion of the first nucleic acid strand to a first securing component prior to said unzipping and
securing a 3′-portion of the second nucleic acid strand to a second securing component prior to said unzipping.

59. The method according to claim 58, wherein said securing is achieved using a nucleic acid bonding technique.

60. The method according to claim 58, wherein said nucleic acid bonding technique is selected from the group consisting of streptavidin/biotin binding and antibody/antigen binding.

61. The method according to claim 55, wherein said unzipping comprises either of the following:
moving the first securing component away from the second securing component, wherein said second securing component is kept relatively stationary;
moving the second securing component away from the first securing component, wherein said second securing component is kept relatively stationary; or
simultaneously moving the first and second securing component away from one another.

62. The method according to claim 58, wherein motion or lack of motion of said first or second securing component is achieved by using a technique selected from the group consisting of optical trapping technology, micropipette technology, viscous drag force, atomic force microscopy, magnetic force microscopy, optical fiber force transducer technology, nano-fabricated cantilever or tip technology, micro-fabricated cantilever or tip technology, nanotube technology, and microelectromechanical technology.

63. The method according to claim 55, wherein the change in force is either an increase or decrease in the force required to unzip the first and second nucleic acid strands of the modified nucleic acid molecule compared to the force required to unzip the nucleic acid molecule.

64. The method according to claim 55, wherein said identifying comprises:
measuring force using force sensor technology.

65. The method according to claim 63, wherein the force sensor technology is selected from the group consisting of optical trapping technology, micropipette technology, viscous drag force, atomic force microscopy, magnetic force microscopy, nano-fabricated cantilever or tip technology, micro-fabricated cantilever or tip technology, nanotube technology, optical fiber force transducer technology, and microelectromechanical technology.

66. The method according to claim 65, wherein the force sensor technology is optical trapping technology comprising a feedback-enhanced optical trap.

67. The method according to claim 55, wherein the binding molecule is selected from the group consisting of a polypeptide, an oligonucleotide, an inorganic chemical compound, an organic chemical compound, and a PNA.

68. The method according to claim 67, wherein the binding molecule is a polypeptide selected from the group consisting of a restriction endonuclease, a polymerase, a helicase, a nuclease, an isomerase, a ligase, activators, repressors, and a histone.

69. The method according to claim 55, wherein said double-stranded nucleic acid molecule is selected from the group consisting of a double-stranded DNA molecule, a double-stranded RNA molecule, and a DNA/RNA duplex molecule.

70. A method of identifying whether a target protein is present in a sample comprising:
providing a sample potentially containing a target protein;
providing a double-stranded nucleic acid molecule comprising first and second nucleic acid strands and having a binding site to which the target protein binds;
contacting the sample and the double-stranded nucleic acid molecule with one another under conditions effective to permit the target protein, if present, to bind to the double-stranded nucleic acid molecule;
unzipping the first and second nucleic acid strands under conditions effective to separate the first and second nucleic acid strands from one another; and
identifying a presence of the target protein by comparing force required to unzip the first and second nucleic acid strands from one another with force required to unzip the double-stranded nucleic acid molecule having no target binding molecule bound thereto, wherein a change in force required to unzip the double-stranded nucleic acid molecule having no target binding molecule indicates the presence of the target protein in the sample.

71. The method according to claim 70 further comprising:
securing a 5′-portion of the first nucleic acid strand to a first securing component prior to said unzipping and
securing a 3′-portion of the second nucleic acid strand to a second securing component prior to said unzipping.

72. The method according to claim 71, wherein said securing is achieved using a nucleic acid bonding technique.

73. The method according to claim 72, wherein said nucleic acid bonding technique is selected from the group consisting of streptavidin/biotin binding and antibody/antigen binding.

74. The method according to claim 71, wherein said unzipping comprises either of the following:
moving the first securing component away from the second securing component, wherein said second securing component is kept relatively stationary;
moving the second securing component away from the first securing component, wherein said second securing component is kept relatively stationary; or
simultaneously moving the first and second securing component away from one another.

75. The method according to claim 74, wherein motion or lack of motion of said first and second securing components is achieved by using a technique selected from the group consisting of optical trapping technology, micropipette technology, viscous drag force, atomic force microscopy, magnetic force microscopy, optical fiber force transducer technology, nano-fabricated cantilever or tip technology, micro-fabricated cantilever or tip technology, nanotube technology, and microelectromechanical technology.

76. The method according to claim 70, wherein said identifying comprises:
measuring force using force sensor technology.

77. The method according to claim 76, wherein the force sensor technology is selected from the group consisting of optical trapping technology, micropipette technology, viscous drag force, atomic force microscopy, magnetic force microscopy, nano-fabricated cantilever or tip technology, micro-fabricated cantilever or tip technology, nanotube technology, optical fiber force transducer technology, and microelectromechanical technology.

78. The method according to claim 77, wherein the force sensor technology is optical trapping technology comprising a feedback-enhanced optical trap.

79. The method according to claim 70, wherein the binding molecule is selected from the group consisting of a polypeptide, an oligonucleotide, an inorganic chemical compound, an organic chemical compound, and a PNA.

80. The method according to claim 79, wherein the binding molecule is a polypeptide selected from the group consisting of a restriction endonuclease, a polymerase, a helicase, a nuclease, an isomerase, a ligase, activators, repressors, and a histone.

81. The method according to claim 70, wherein said double-stranded nucleic acid molecule is selected from the group consisting of a double-stranded DNA molecule, a double-stranded RNA molecule, and a DNA/RNA duplex molecule.
